(19) United States
(12) Patent Application Publication
(10) Pub. No.: US 2009/0054328 A1
(43) Pub. Date: Feb. 26, 2009

(54) Reagents and Methods for Modulating Gene Expression Related to Hypertension

(75) Inventors: Primal de Lanerolle, Oak Park, IL (US); Yoo Jeong Han, Skokie, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Board of Trustees of the University of Illinois, Urbana, IL (US)

(21) Appl. No.: 12/088,390

(22) PCT Filed: Oct. 20, 2006
(86) PCT No.: PCT/US06/60121

§ 371 Date: Jul. 25, 2008

Related U.S. Application Data
(60) Provisional application No. 60/728,965, filed on Oct. 21, 2005.

(51) Int. Cl. A61K 38/00; C12N 15/00; C12N 5/06; A61P 9/00; C12Q 1/68

(52) U.S. Cl. 514/12; 435/320.1; 435/325; 435/6; 435/375

(57) ABSTRACT

This invention relates to gene expression and proliferation in eukaryotic, preferably mammalian and most preferably human cells. The invention specifically relates to hypertension associated with proliferation and contractility of vascular smooth muscle cells.

This application claims priority to U.S. provisional patent application, Ser. No. 60/728,965, filed Oct. 21, 2005, the disclosure of which is explicitly incorporated by reference herein.

This invention was made with government support under grant HL 59618 and HL64702 by the National Institutes of Health. The government has certain rights in the invention.

### BACKGROUND OF THE INVENTION

1. Field of the Invention

This invention relates to gene expression and proliferation in eukaryotic, preferably mammalian and most preferably human cells. The invention specifically relates to hypertension associated with proliferation and contractility of vascular smooth muscle cells. The invention particularly provides methods and reagents for detecting, evaluating, diagnosing, monitoring and treating hypertension and proliferative disorders, as well as screening methods for identifying molecules capable of reducing hypertension or proliferative disorders in an animal and reagents useful therewith,

2. Background of the Related Art

Cell growth, proliferation and migration are required for embryonic development, organogenesis, immune cell functions, wound healing and other cellular functions. They are also hallmarks of many diseases. In the vasculature, the proliferation and migration of vascular smooth muscle cells (VSMC) contribute to important vascular diseases such as atherosclerosis and intimal hyperplasia (Chen et al., 2004, Nat Cell Biol. 6: 872-883). This is also true in hypertension, which is characterized by increased VSMC contraction and vascular remodeling. Vascular remodeling results from the growth, proliferation and migration of VSMC within blood vessels. This, in turn, leads to thickening of the vessel wall, a decrease in vessel caliber and an increase in resistance to blood flow (Lifton et al., 2001, Cell 104: 545-556),

Vascular remodeling, like all types of cell proliferation, requires both gene expression and profound changes in the cytoslceleton. Cells have to grow and duplicate their contents before they can divide and the physical process of cell division requires an acto-myosin II dependent contractile event (Alberts et al., 2002, Molecular Biology of the Cell, 4th Ed. (New York: Garland Science)). How target gene transcription and cytoskeletal dynamics are coordinately regulated and the signaling pathways that exercise this regulation are not clear,

VSMC contraction is regulated by the calcium-dependent phosphorylation of myosin II light chains (MLC-P) by myosin light chain kinase (MLCK; de Lanerolle & Paul, 1991, Am. J. Physiol. 261: L1-14). The MLCK gene is a single copy gene located on chromosome 3q21 (Potier et al., 1995, Genomics 29: 562-570) that encodes 3 proteins (Lazar & Garcia, 1999, Genomics 57: 256-267): non-muscle MLCK (210 kDa), smooth muscle MLCK (130 kDa) and telokin (20 kDa). In the chicken, the translation start sites for non-muscle MLCK, smooth muscle MLCK and telokin are in exons 1, 15 and 29, respectively (Birukov el al., 1998, J. Cell. Biochem. 70: 402-413) and the expression of each protein appears to be independently regulated by separate promoters (Wainwright et al., 2003, Proc Natl Acad Sci U S A. 100: 6233-6238). Only the telokin promoter has been identified and it is embedded in the intron immediately proceeding exon 29 of the avian MLCK gene (Gallagher & Herring, 1991, J Biol. Chem. 266: 23945-23952). However, differential expression, and the location of sequences that mediate such expression, is unknown in humans and is thus an impediment to understanding how MLCK and MLC-P mediate normal and pathological states in humans associated with disease,

MLCK activity (and MLC-P levels) are modulated in response to cellular signals. The small G protein (GTPase) Ras regulates cellular responses by affecting cytoskeletal dynamics and the transcription of target molecules (Etienne-Manneville & Hall, 2002, Nature 420: 629-635; Chien & Hoshijima, 2002, Nat Cell Biol. 6: 807-808). Ras is activated by mitogenic stimuli (Alberts et al., 2002, Id.) and Ras mutations are common in transformed cells (Malumbres & Barbacid, 2003, Nat Rev Cancer, 3: 459-465), Ras, via phosphorylation and activation of ERK (Chien & Hoshijima, 2002, Id.), stimulates transcription and drives progression through the cell cycle, in part, by activating cyclin-dependent kinases (Peeper et al., 1997, Nature 386: 177-181). Ras also regulates cell motility via ERK, which phosphorylates and stimulates MLCK activity and, hence, increases MLC-P (Klemke et al., 1997, J. Cell Biol. 137: 481-492). Ras has been implicated in a variety of cardiovascular diseases (Chien & Hoshijima, 2002, Id.) and Ras appears to play a direct role in regulating VSMC proliferation: an important regulator of VSMC proliferation, hyperplasia suppressor gene (HSG), induces cell cycle arrest by inhibiting Ras/MEK/ERK signaling (Chen et al., 2004, Id). Other experiments have shown that Ras, via SRF, regulates the expression of genes involved in both the proliferation and differentiation of VSMC (Wang & Olson. 2004, Curr Opin Genet Dev. 14: 558-566).

Animal models of hypertension, specifically spontaneously hypertensive rats (SHR) and normotensive Wistar-Kyoto (WKY) rats, provide a means for investigating the molecular mechanisms that regulate the expression of MLCK in VSMC. SHR animals are a well-established model of hypertension (Yamori, 1984, in Handbook of Hypertension, vol. 4, (DeJong, ed.), New York: Elsevier, pp. 224-239) in which an increase in blood pressure involves increases in VSMC contractility and proliferation (Lifton el al., 2001, Id.). These two processes work together to increase vascular resistance by decreasing the caliber of blood vessels (Touyz, 2003, Curr Hypertem Rep, 5: 155-164). In addition, VSMC from SHR have increased rates of cell proliferation compared to VSMC from normotensive rats (Chen el al., 2004, Id.). Coupled with a central role for Ras in VSMC proliferation (Etienne-Manneville & Hall, 2002, Id.), VSMC from SHR constitute an effective experimental system for investigating how GTPases regulate both cytoskeletal dynamics and gene regulation.

The molecular mechanism(s) by which GTPases coordinately regulate cytoskeletal dynamics and expression of target proteins required for cell division is largely unknown, and thus there is a need in the art to determine the molecular mechanisms involved in these processes, particularly as they relate to human diseases such as hypertension.

### SUMMARY OF THE INVENTION

This invention provides methods and reagents for detecting, evaluating, diagnosing, monitoring and treating hypertension, as well as screening methods for identifying molecules capable of reducing hypertension in an animal. The invention also provides methods and reagents for detecting, evaluating, diagnosing, monitoring and treating cellular proliferation and proliferative disorders in a patient.

In one aspect, the invention provides a recombinant expression construct comprising an inducible promoter, wherein the inducible promoter comprises a promoter from a mammalian myosin light chain kinase (MLCK) bearing one or a plurality of mutations that increase transcription from the promoter in the presence of a transcription factor produced in the cell after stimulation of a cellular signaling pathway comprising a Ras oncogene.

In another aspect, the invention provides methods for identifying a compound that induces gene expression from a recombinant expression construct as provided herein, comprising the steps of: a) contacting a recombinant mammalian cell comprising said recombinant expression construct with the compound; b) comparing gene expression from the recombinant expression construct in the presence and absence of the compound; and c) identifying a compound that induced expression from the recombinant expression construct when gene expression is higher in the presence than in the absence of the compound.

The invention also provides methods for identifying a compound that decreases angiotensin-induced gene expression from a recombinant expression construct according to claim 1, comprising the steps of: a) contacting a recombinant mammalian cell comprising said recombinant expression construct with the compound in tire presence and absence of angiotensin; b) comparing gene expression from the recombinant expression construct in the presence and absence of the compound; and c) identifying a compound that decreases angiotensin-induced gene expression from a recombinant expression construct when gene expression is lower in the presence than in the absence of the compound.

Specific preferred embodiments of the present invention will become evident from the following more detailed description of certain preferred embodiments and the claims.

### BRIEF DESCRIPTION OF THE DRAWINGS

FIGS. 1A through 1C disclose the results of Example 1 on isolation and analysis of intron 14-15 of the rat MLCK gene. FIG. 1A shows the results of polymerase chain reaction (PCR) amplification of intron 14-15: PGR primers (SEQ ID NO: 5 in exon 14; SEQ ID NO: 6 in exon 15) based on the rat myosin light chain kinase (MLCK) gene were used to amplify intron 14-15 from genomic DNA obtained from SHR and WKY rats. The translation start site of smooth muscle MLCK in exon 15 is shown (ATG). FIG. 1B shows a comparison between DNA sequences of intron 14-15 from SHR (SEQ ID NO: 7) and WKY (SEQ ID NO: 8) rats. This comparison revealed the presence of a 12 bp insertion in the SHR sequence (in boldface) not found in the WKY sequence, WKY also contains 3 different single nucleotide polymorphisms (underlined) compared to SHR. The transcription initiation site (+1) of smMLCK was identified using 5′-RACE. Analysis of transcription factor binding sites using the Transcription Element Search System (TESS) demonstrated the presence of a TATA box and serum response factor binding site or CArG box (uppercase). FIG. 1C shows the results of analysis of intron 14-15in other normotensive and hypertensive rat strains. Stroke-prone SHR (SHRSP; SEQ ID NO: 10), a closely related genetic strain of SHR (Yamori, 1984, Id.), also contains the 12 bp insertion while normotensive Sprague-Dawley (SD; SEQ ID NO: 9) and WKY rats do not. The sequence shown for WKY is SEQ ID NO: 9 and the sequence shown for SHR is SEQ ID NO: 10.

FIGS. 2A through 2C show the results of electromobility shift assay (EMSA) and chromatin immunoprecipitation (ChIP) analyses of intron 14-15. FIG. 2A shows experimental evidence that TATA binding proteins (TBP) binds to intron 14-15 from SHR animals. Purified TBP was incubated with ³²P-labeled intron 14-15 from SHR and analyzed using EMSA. TBP induced a concentration-dependent appearance of a slower migrating band (arrow). A 10-fold excess of cold competitor nucleic acid eliminated detection of this band. FIG. 2B shows increased serum responsive factor (SRF) binding to the CArG box in the SHR promoter. Two oligonucleotides (box) representing regions of the MLCK promoters containing the CT repeats and CArG box were synthesized (SEQ ID NO: 11 and SEQ ID NO: 12). The only difference between them is the presence of a 12 bp sequence (red) in the SHR oligonucleotides. Nuclear extracts were isolated from cells expressing SRF (NE-SRF). Four different concentrations of the extracts were incubated with ³²P-labeled WKY oligonucleotides (SEQ ID NO: 11) or SHR oligonucleotides (SEQ ID NO: 12). Autoradiography demonstrated the presence of slower migrating bands (arrow) that increased in intensity as the concentration of the extracts was increased. These bands were more intense when incubated with the oligonucleotides representing the SHR promoter. Cold competitor abolished this band (lane 5 & 12) whereas the SRF antibodies supershifted this band (lane 11, star). FIG. 2C shows the results of ChIP assays performed with antibodies to SRF or RNA polymerase II. Non-specific IgG was used as a negative control (Con IgG). Input chromatin (0.2, 0.02 and 0,002%) and immunoprecipitated DNA (4, 0.4 and 0,04%) were amplified with primers specific for the β-globin promoter or intron 14-15 of MLCK gene. SRF and RNA polymerase II are not present in the β-globin promoter. However, SRF and RNA polymerase II bound to intron 14-15 of the MLCK gene in VSMC from WKY rats. FIG. 2D shows the results of ChIP assays performed with cross-linked chromatin from VSMC of SHR or WKY rats that was immunoprecipilated with antibodies to SRF. Input chromatin (0.1, 0.01 and 0.001%) and immunoprecipitated DNA (2, 0.2, and 0.02%) were amplified with primers specific for the intron 14-15. Comparing the relative abundance of the signals (as described in Example 1) confirmed increased binding of SRF to the intron in SHR compared to the intron in WKY cells.

FIGS. 3A and 3B show the results of reporter gene expression under the control of the MLCK promoter in intron 14-15 from SHR. FIG. 3A shows the results of promoter activity from MLCK intron 14-15 from WKY (W-pMK), Sprague-Dawley (SD-pMK) and SHR (S-pMK) rats analyzed using a luciferase reporter gene assay. These results demonstrated increased responsiveness to SRF in the intron 14-15 from SHR. While SRF co-expression increased the activities of all 3 promoters, it increased SHR promoter activity much more than WKY and SD promoter activity. When transfected with 50 ng of a plasmid encoding SRF (see Example 3 below), promoter activity increased 2.7-fold (WKY), 2.1-fold (SD) and 7.2-fold (SHR), compared to control cells transfected with 50 ng pcDNA 3.1 (negative control plasmid). This experiment was repeated 5 times and the means +/− SE are shown. *P<0.05 compared to W-pMK plus 50 ng SRF and **P<0.05compared to W-pMK plus 150 ng SRF. FIG. 3B shows that increased SRF responsiveness is due to the presence of the 12 bp insertion in the intron 14-15 promoter. The CT repeats from SHR rats was inserted into the intron 14-15 promoter from WKY rats (WS-pMK, shown schematically). Luciferase activity assays on cells transfected with W-pMK, S-pMK or WS-pMK, with or without SRF co-expression, showed that the 12 bp sequence increased the activity of the WKY promoter to the same level as the SHR promoter. *P<0.05 compared to the activity of W-pMK plus SRF.

FIGS. 4A through 4D shows the results of Ras regulation of MLCK expression via SRF. FIG. 4A illustrates that dominant negative SRF down-regulates SRF and MLCK expression. VSMC from WKY rats were infected with adenoviruses expressing a short form of SRF (AdSRF-S), which acts as a dominant negative repressor of the endogenous SRF (Davis et al., 2002, Am J Physiol Heart Circ Physiol 282: H1521-33), or a green fluorescent protein (GFP)-encoding adenoviral construct (AdGFP) as a control. Western blot analyses were performed using commercially available antibodies to full length SRF (SRF-FL) (Upstate Biotechnology), MLCK and actin (loading control). FIG. 4B shows the results of experiments in which VSMC were co-transfected with SRF and plasmids expressing dominant negative Ras or dominant negative Rho. The cells were extracted and analyzed by Western blotting using antibodies to MLCK. SRF (lane 2) increased MLCK expression compared to control VSMC transfected with pcDNA 3.1 (negative control plasmid; lane 1). Dominant negative Ras blocked this increase in expression while dominant negative Rho had no effect. The experiments in FIGS. 4A and 4B were repeated 3 times and the mean changes in density of the bands, compared to control (1 in each panel), are shown. Actin was used as a loading control in both panels. FIG. 4C shows the results of reporter gene assays performed in the presence or absence of dominant negative Ras. VSMC from SHR were transiently transfected with empty vector (pcDNA) or N17Ras and luciferase assays were performed as described in the Examples below. N17Ras directly inhibits the luciferase activity of intron 14-15 from SHR. This experiment was repeated 3 times and the means +/− SE are shown (*P<0.05 compared to the activity of S-pMK plus pcDNA). Northern blot analyses further showed that N17Ras decreased MLCK mRNA expression in VSMC from SHR (inset). FIG. 4D shows the results of antisense inhibition of ERK expression and its effect on MLCK expression in SHR. VSMC from SHR were transiently transfected with vehicle (veh only), scrambled oligonucleotides (Con) or antisense (AS) oligonucleotides to ERK. Un-P-MLC and P-MLC indicate unphosphorylated and monophosphorylated forms of MLC₂₀. The experiment in FIG. 4D was repeated 4 times and a representative blot is shown.

FIGS. 5A through 5D show that ERK-P, MLCK and MLC-P are up-regulated in SHR. FIG. 5A shows phosphorylated ERK (ERK-P) in blood vessels. Western blot analyses were performed on blood vessels removed from SHR and WKY rats of differing ages using antibodies to ERK-P and actin (loading control). FIG. 5B shows the results of Northern blot analysis on the level of MLCK mRNA in blood vessels from 14 week old SHR and age-matched WKY rats. 18S rRNA was used as a loading control. FIG. 5C shows the results of Western blot analyses using antibodies to MLCK or actin (loading control) on blood vessels removed from SHR and WKY rats. FIG. 5D shows quantification of phosphorylated MLC (MLC-P) in blood vessels removed from SHR and WKY rats of differing ages. The stoichiometry of MLC-P (bottom) was calculated following urea/glycerol gel-immunoblotiing (top). Un-P-MLC and P-MLC indicate un- and mono-phosphorytated forms of MLC₂₀. Each experiment was repeated at least 3 times and mean±SE are shown in each bar graph. *P<0.01 compared to age-matched WKY rats.

FIGS. 6A through 6C show in vivo effects of inhibiting MEK on blood pressure, signaling molecules and vascular remodeling. FIG. 6A shows the results of continuous 3-week administration of DMSO or U0126 to SHR that were 8 weeks old at the start of the experiment. Systolic blood pressure (SBP) was measured every 5 days. Values are mean±SE, n=6, *SHR+U0126 vs. SHR+DMSO, P<0.0001. FIG. 6B shows the results of analysis of signaling molecules in the aortas from U0126-treated SHR, demonstrating lower levels of ERK-P, MLCK expression and MLC-P compared to control. No significant differences were found in RhoA expression or phosphorylated myosin phosphorylase-1 (MPasel-P) after U0126treatment. Actin was used as a loading control. FIG. 6C shows histological analysis of the medial layer of aortas and mesenteric arteries from SHR treated with DMSO or U0126. The data from representative experiments (n=4) are shown in FIGS. 6B and 6C.

FIG. 7 shows in vivo effects of inhibiting MEK on blood pressure in 16 week old SHR or age-matched WKY rats. DMSO or U0126 was continuously administered for 3 weeks to SHR or WKY Rats. Systolic blood pressure was measured every 5 days. Values are mean ±SE, 48 n=6, *SHR+U0126 vs. SHR+DMSO, P<0.001.

FIG. 8 shows in vivo effects of inhibiting MLCK on blood pressure in SHR. DMSO or ML-7 (a specific inhibitor of MLCK; Fazal et al, 2005, Mol. Cell. Biol. 25:6259-6266), was continuously administered for 3 weeks to adult SHR. Systolic blood pressure was measured every 5 days. Values are mean±SE, n=4, *SHR+ML-7 vs. SHR+DMSO, P<0.001.

Statistical Analysis. Results are expressed as mean±SE. The data were analyzed using an unmatched Student's T-test and One-Way ANOVA (SigmaStat, Systat, Point Richmond, Calif.). P<0.05 were considered statistically significant.

FIGS. 9A and 9B show that ML-7 induces apoptosis in mammary and prostate cancer cells. FIG. 9A shows that ML-7 induces apoptosis in Mm5MT mammary cancer cells and FIG. 9B MLL prostate cancer cells. Mm5MT or MLL cells were treated with vehicle (0) or increasing concentrations (5-25 μM) of ML-7 for 16 h. The cells were collected and apoptosis was quantified by FACS analysis. The annexin V and PI positive cells as a percent of total cells, at each concentration of ML-7, are shown (N=4). *P<0.05 compared to control.

FIG. 10 shows the chcraoprcventive effect of ML-7 in mouse mammary gland organ culture. Mammary glands from Balb/c mice were treated as described below and the effects of etoposide and ML-7 on preventing MAL formation was quantified. ML-7 prevents MAL formation at 0.1 μM while a similar level of inhibition requires a 10× higher concentration of etoposide. Percentage inhibition was calculated by comparing the incidence in the control glands with the treated groups. Results were subjected to χ² analysis. *P<0.05 compared to control.

FIG. 11 shows that ML-7 stimulates the ability of etoposide to induce apoptosis in Mm5MT cells in vitro. Mm5MT cells were pretreated with vehicle (open bars) or 10 μM ML-7 (stippled bars) for 2 hours prior to adding the indicated concentrations of etoposide. Cells were collected 16 hours after adding etoposide and apoptosis was quantified by FACS analysis. The annexin V and PI positive cells as a percentage of total cells, at each concentration of etoposide, are shown (N=4, *P<0.05, **P<0.001 vs. etoposide alone). (Inset) Mm5MT cells were treated with vehicle (control), 10 μM ML-7, 30 μM etoposide or 10 μM ML-7 and 30 μM etoposide. MLC-P was measured by urea/glycerol gel-immunoblotting. In the inset, Un and P identify unphosphorylated and phosphorylated MLC20, respectively. Note the decrease in the phosphorylated band in the treated groups compared to the control. This experiment was repeated four times and the data from a representative experiment is shown.

FIGS. 12A and 12B show that ML-7 and etoposide have a potent, additive tumouricidal effect on mammary tumors. Female MMTV/C3H/HeN mice were inoculated with Mm5MT cells as described in Methods. Drug treatment was started 7 days later when the mice had developed palpable tumours. The mice were sacrificed after 28 days of drug treatment. FIG. 12A shows tumors removed from representative mice in each treatment group and a ruler is included as a size reference. FIG. 12B shows the means±SE for tumour weight in each group (N=5, *P<0.05, **P<0.001 vs. vehicle control, +P<0.05 vs, etoposide alone).

FIGS. 13A and 13B show that ML-7 and etoposide synergize to enhance tumor necrosis in vivo. FIG. 13A shows a graph of data from photomicrographs analyzed in blinded fashion for areas of necrotic and viable tumour and quantified using manual tracing tools within MetaMorph 6.2. Only the tumors from mice treated with both etoposide and ML-7 showed a significant decrease in viable tumor area compared to control (N=5, *P<0.05). FIG. 13B shows representative medium power images of tumors from control, etoposide-treated, ML-7-treated, and etoposide plus ML-7-treated mice, as indicated. Limited areas of necrosis are present in tumors from etoposide- and ML-7-treated mice (arrow), but adjacent tumor is viable and mitotic figures are easily found. In contrast, larger areas of necrosis are present in tumors from etoposide plus ML-7-treated mice (arrow) and adjacent viable tumor shows signs of impending apoptosis, including incohesion (asterisk). Bar=100 μm.

FIG. 14 shows that ML-7 stimulates the ability of etoposide to induce apoptosis in MLL cells in vitro, MLL cells were pretreated with vehicle (open bars) or 5 μM ML-7 (stippled bars) for 2 hours prior to adding the indicated concentrations of etoposide. Ceils were collected 16 hours after adding etoposide and apoptosis was quantified by FACS analysis. The annexin V and PI positive cells as a percentage of total cells, at each concentration of etoposide, are shown (N=4, *P<0.05 and **P<0.01 vs. etoposide alone), (Inset) MLL cells were treated with vehicle (control), 5 μM ML-7, 30 μM etoposide or 5 μM ML-7 and 30 μM etoposide. MLC-P was measured by urea/glycerol gel-immunoblotting. Un and P identify unphosphorylated and phosphorylated MLC20, respectively. ML-7 alone and with 30 μM etoposide, resulted in a substantial decrease in the phosphorylated MLC20 band where as etoposide resulted in a smaller decrease in MLC-P. This experiment was repeated four times and the data from a representative experiment is shown.

FIGS. 15A and 15B show ML-7 and etoposide have a potent, additive tumoricidal effect on prostate tumors. FIG. 15A shows pictures of tumors removed from representative rats in each treatment and FIG. 15B shows the means±SE for tumor weight in each group (N=5, *P<0.05, **P<0.001 vs. vehicle control, +P<0.05 vs. etoposide alone).

### DETAILED DESCRIPTION OF THE PREFERRED EMBODIMENTS

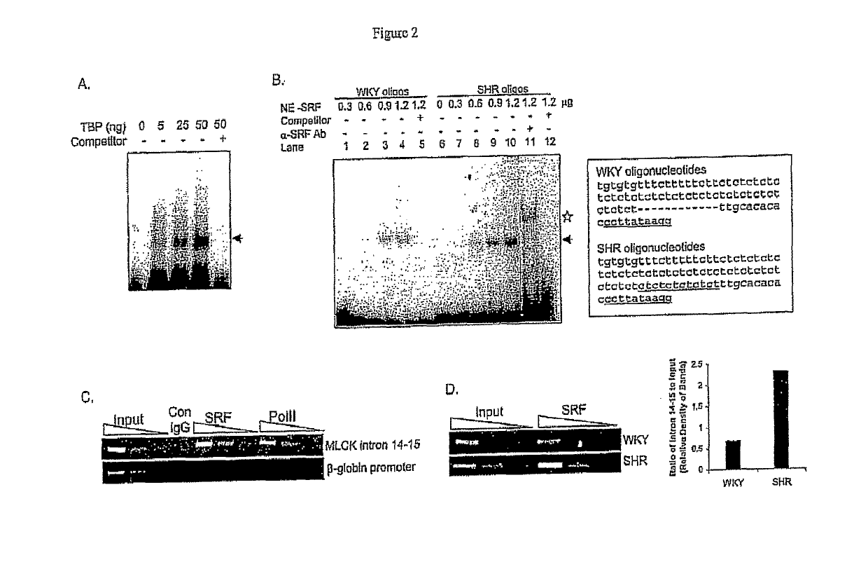

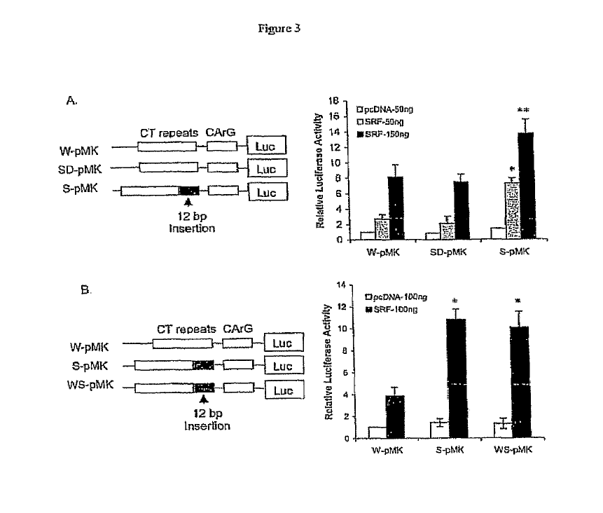

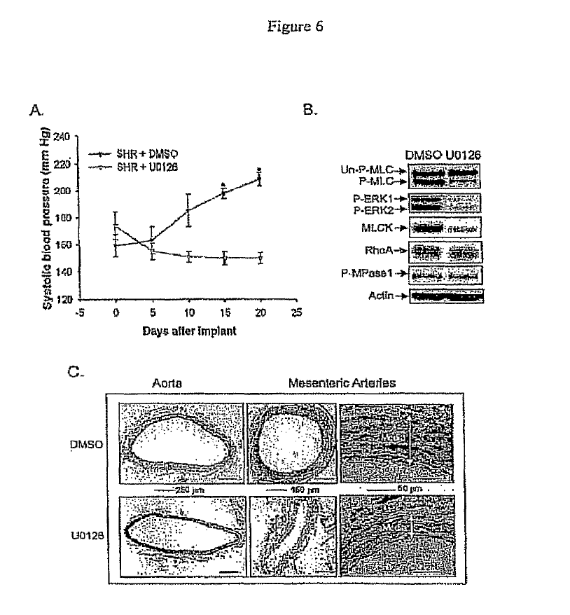

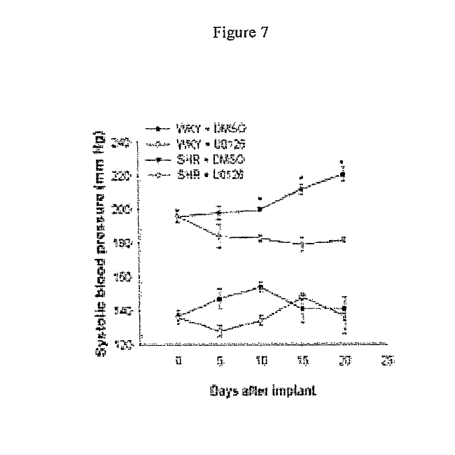

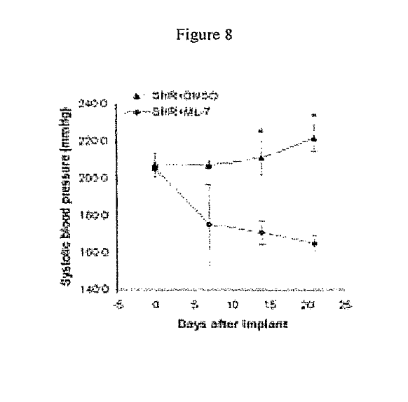

As shown for the first time herein, MLCK expression is regulated by Ras signaling via SRF. Reporter gene assays (FIGS. 3A through 3C) performed with a rat model of hypertension (SHR) showed that increase in MLCK expression is due to the presence of an insertion mutation that is in close proximity to a SRF binding site (FIG. 2A). This insertion mutation results in up-regulation of smooth muscle MLCK expression in SHR, specifically in intron 14-15 of the rat MLCK gene containing promoter elements, included a TATA box and multiple Ras responsive elements. The SHR promoter also contains an insertion mutation that is proximal to an SRF binding site and mediates increased promoter responsiveness to SRF. In addition, the physiological significance of these observations were demonstrated in that ERK-P, MLCK expression and MLC-P are increased in SHR, and ERK-P, MLCK mRNA and protein levels and MLC-P also increase with age in SHR. Further, these increases roughly parallel the increase in blood pressure characteristic of this animal's phenotype. These data establish an important role for the MLCK/MLC-P pathway in the pathophysiology of hypertension and suggest that inhibiting the pathway could be useful in treating hypertension. Blocking Ras signaling in vitro using dominant-negative Ras mutants, antisense oligonucleotides or a MEK inhibitor (U0126) decreased MLCK expression in VSMC, In vivo experiments showed that inhibiting MEK, a member of the Ras gene regulatory cascade, blocked ERK-P, MLCK expression and MLC-P and decreased vascular remodeling and blood pressure (FIGS. 6A through 6C and FIG. 7). Also, in vivo experiments showed that inhibiting MLCK activity using ML-7, a specific inhibitor of MLCK (Fazal et al, 2005, Mol. Cell Biol. 25:6259-6266), decreased blood pressure (FIG. 8). These data established that regulating MLCK expression, which itself regulates cytoskeletal dynamics and contractile events associated with cell division, is part of the genetic program that results in cell proliferation.

In vitro experiments showed that blocking Ras signaling using a variety of approaches inhibited ERK activation, MLCK expression and MLC-P. Moreover, in vivo experiments demonstrated that inhibiting MEK decreased ERK-P, MLCK expression, MLC-P, vascular remodeling and blood pressure.

These data established the importance of increased MLCK expression in the pathophysiology of hypertension and suggest that it could be important in other proliferative disorders. In this context, it is interesting that MLCK expression is stimulated by SRF. SRF was first identified as an activator of the c-fos promoter (Norman et al., 1988, Cell 55: 989-1003). C-fos is an early intermediate gene that stimulates proliferation of various cell types (Arsenian et al., 1998, EMBO J. 17: 6289-6299). As reviewed by Wang and Olson (2004, Curr Opin Genet Dev. 14: 558-566), Ras activates ERK, active ERK (ERK-P) phosphorylates ternary complex factors (TCP) of the ETS-domain family and, eventually, phospho-TCF/SRF complexes bind to and turn on target genes.

That Ras regulates MLCK expression may be especially important because Ras mutations have been identified in many human cancers (Malumbres & Barbacid, 2003, Nat Rev Cancer, 3: 459-465). Ras also affects MLC-P because phosphorylation by ERK increases MLCK activity (Klemke el al., 1997, J. Cell Biol. 137: 481-492). Moreover, MLC-P and MLCK appear to be involved in determining cell fate. The expression of myosin II heavy chain in proliferating VSMC is regulated by growth factors (Wang et al., 2004, Nature 428: 185-189) and myosin II has been identified in an epidermal growth factor receptor induced, transformation specific signaling module (McManus et al., 2000, J Biol Chem. 275: 35328-35334). Myosin II phosphorylation by cdc2 kinase (Satterwhife el al., 1992, J Cell Biol. 118: 595-605; Yamakita et al., 1994, J Cell Biol. 124: 129-37) has been implicated in regulating the timing of mitosis, and increasing MLC-P prolongs the cell cycle (Cai et al., 1998, Am J Physiol. 275: C1349-1356). In addition, MLCK has been localized in the cleavage furrow of mammalian cells (Chew et al., 2002J Cell Biol. 156: 543-553) and knocking out myosin II expression results in a defect in cytokinesis (De Lozanne & Spudich, 1987, Science 236: 1086-1091), further supporting a role for MLCK/MLC-P in cytokinesis. Other experiments have shown that inhibiting MLCK induces apoptosis (Fazal et al., 2005, Mol. Cell. Biology, 25: 6259-6266). Lastly, the data in FIG. 6 show that inhibiting MEK decreases MLCK expression, MLC-P and vascular remodeling. Although the effects of U0126 may be pleiotrophic, the data in FIG. 6, especially in view of the above discussion, strongly support the idea that MLCK via MLC-P is important in determining cell fate and that stimulating MLCK expression is part of the genetic program induced by Ras that results in cell proliferation.

Transcriptional activity of SRF is also regulated by Rho (Miralles et al., 2003, Cell 113: 329-342). In light of the importance of Rho in regulating VSMC contractility (Kimura et ah, 1996, Science 273: 245-248; Uehata et al, 1997, Nature 389: 990-994), one would predict that Rho would also regulate MLCK expression. However, (surprisingly) dominant-negative Rho did not decrease the SRF-stimulated increase in MLCK expression in VSMC in vitro (FIG. 3B) and Rho A levels and the phosphorylation of MPasel were unchanged (FIG. 6A) by U0126 in vivo.

These results were unexpected because, inter alia, Rho A expression is increased in hypertension (Seasholtz et al., 2001, Circ. Res. 89: 488-495) and inhibiting Rho kinase decreases blood pressure in SHR (Uehata et al., 1997, Nature 389: 990-994). Mechanistically, Rho kinase phosphorylates and inactivates myosin phosphatace-1 (MPasel; Kimura et al., 1996, Science 273: 245-248) and inhibiting Rho kinase maintains MPasel in the active form, thereby apparently decreasing the level of MLC-P (Uehata et al, 1997, Nature 389: 990-994). Although Rho A expression was increased in SHR compared to WKY rats, no changes were found in Rho A levels or the phosphorylation of MPasel in response to U0126 treatment, and dominant-negative Rho did not affect MLCK expression (FIG. 3B). While previous data have demonstrated the importance of Rho signaling (Touyz & Schiffrin, 1997, J. Hypertens. 15: 1431-1439), these data demonstrated that Ras signaling is also important in hypertension and emphasize the complexity of the signaling pathways involved in the development of hypertension.

Thus, in certain embodiment, the invention provides methods for reducing blood pressure in a patient, preferably a patient that has hypertension. As used herein, the term “patient” includes human and animal subjects. In one embodiment, the methods comprise the step of inhibiting MEK activity in the patient. In another embodiment, the methods comprise the step of inhibiting MLCK activity in the patient. Methods for inhibiting MEK activity in a patient are described herein. For example, MEK activity can be inhibited using dominant-negative Ras mutants, antisense oligonucleotides, or MEK inhibitors as described herein. Additional examples of MEK inhibitors that may be used in methods of the invention include, but are not limited to, those disclosed in U.S. Pat. Nos. 7,030,119, 7,001,905, 6,835,749, 6,750,217, 6,703,420, 6,696,440, 6,638,945, 6,506,798, 6,469,004, 6,455,582, 6,440,966, and 6,310,060, all of which are incorporated herein by reference. Examples of MLCK inhibitors that may be used in the methods of the invention include, but are not limited to, those described herein and those disclosed in U.S. Pat. No. 4,943,581 and US Patent Application Publication No. 20050261196, both of which are incorporated herein by reference.

As discussed above and as demonstrated in the Examples below, inhibiting MLCK can induce apoptosis in cancer cells and can inhibit tumor growth (i.e. tumor cell proliferation). Thus, the invention provides methods for treating conditions associated with increased cell proliferation. As used herein, “treatment” or “treat” refers to both therapeutic treatment and prophylactic or preventative measures. Those in need of treatment include those already with the disorder as well as those prone to have the disorder or those in which the disorder is to be prevented.

In certain embodiments, the invention provides methods for inhibiting cell proliferation, including tumor cell proliferation and vascular smooth muscle cell proliferation, by contacting the cell with an MLCK inhibitor. In certain embodiments, the invention provides methods for treating or preventing tumor cell growth comprising administering an effective amount of a MLCK inhibitor to a patient in need thereof.

In other embodiments, the invention provides methods for inhibiting cell proliferation, including tumor cell proliferation, by contacting the cell with an MLCK inhibitor in combination with a chemotherapeutic agent. In certain embodiments, the invention provides methods for treating or preventing tumor cell growth comprising administering an effective amount of a MLCK inhibitor in combination with a chemotherapeutic agent to a patient in need thereof. The MLCK inhibitor can be administered to the patient before or after administering the chemotherapeutic agent, or simultaneously with the chemotherapeutic agent.

Chemotherapeutic agents are known in the art, and include, for example, cis-platin, paclitaxel, carboplatin, etoposide, hexamethylamine, melphalan, and anthracyclines. In a particular embodiment, the chemotherapeutic agent is etoposide or cisplatin. For example, cell proliferation can be inhibited in a patient having breast or prostate cancer by administering to the patient a combination of an MLCK inhibitor and etoposide, or cell proliferation can be inhibited in a patient having lung cancer by administering to the patient a combination of an MLCK inhibitor and cisplatin. Those of skill in the art can readily determine appropriate chemotherapeutic agents to use in combination with the MLCK inhibitor based on the type of condition affecting the patient.

The invention also provides pharmaceutical compositions comprising a compound identified in a method of the invention, an inhibitor of MLCK as described herein, or an inhibitor of MEK activity as described herein.

The term “pharmaceutical composition” as used herein refers to a composition comprising a pharmaceutical acceptable carrier, excipient, or diluent and a chemical compound, peptide, or composition as described herein that is capable of inducing a desired therapeutic effect when properly administered to a patient

The term “therapeutically effective amount” refers to the amount of growth hormone or a pharmaceutical composition of the invention or a compound identified in a screening method of the invention determined to produce a therapeutic response in a mammal. Such therapeutically effective amounts are readily ascertained by one of ordinary skill in the art and using methods as described herein.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company,

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

Dosage levels of the order of from about 0.1 mg to about 140 mg per kilogram of body weight per day are useful in the treatment of patients according to the methods of the invention (about 0.5 mg to about 14 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water,

Dosing frequency will depend upon the pharmacokinetic parameters of the particular inhibitor used in the formulation. Typically, a clinician administers the composition until a dosage is reached that achieves the desired effect The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

In certain embodiments, the invention provides methods for diagnosing a proliferative disorder, comprising assaying MLCK expression in a patient sample, wherein overexpression of MLCK compared with expression of MLCK in a control sample indicates a proliferative disorder. In other embodiments, the invention provides methods for diagnosing a proliferative disorder, comprising detecting mutations in an MLCK promoter in a patient sample. In one embodiment, the mutation comprises a 12 basepair insertion having the sequence CTCTCTCTCTCT (SEQ ID NO: 1) inserted proximal to a CArG site. The proliferative disorder can be, for example, hypertension, cancer, or benign tumor growth.

The invention further provides a recombinant expression construct comprising an inducible promoter, wherein the inducible promoter comprises a promoter from a mammalian myosin light chain kinase (MLCK) bearing one or a plurality of mutations that increase transcription from the promoter in the presence of a transcription factor produced in the cell by stimulation of a signaling pathway comprising a Ras oncogene.

In one embodiment, the promoter of the recombinant expression construct is the MLCK promoter from a rat expressing the SHR phenotype. In another embodiment, the recombinant expression construct comprises a 12 basepair insertion having the sequence CTCTCTCTCTCT (SEQ ID NO: 1) inserted proximal to a CArG site. Gene expression in a recombinant cell comprising the construct can be induced, for example, by contacting the cell with angiotensin. Angiotensin can induce expression of MLCK.

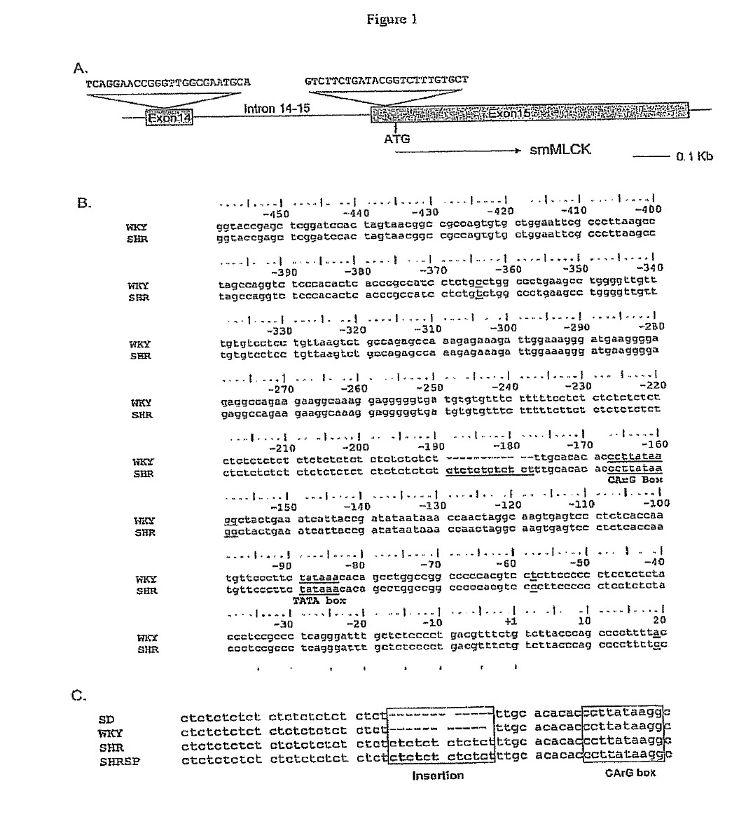

Inducible promoters as provided by this invention are exemplified by the MLCK promoter from intron 14-15 in the MLCK gene in SHR rats. However, the invention also provides inducible promoters comprising sequences other than the exemplary MLCK promoter described in the Examples below and FIG. 1, wherein the position of the TATA box, the CArG element, and the 12 bp CTCTCTCTCTCT (SEQ ID NO: 1) insertion are arranged in substantially the same relative position to one another to provide an inducible promoter as set forth herein.

In certain embodiments, the recombinant expression constructs of the invention are useful for providing regulated, either inducible or repressible, expression of genes, preferably mammalian genes and most preferably genes for which modulation of expression provides a benefit, either in vitro (such as in maximizing the production of a recombinant protein) or in vivo (most preferably MLCK).

The recombinant expression constructs of the invention are also advantageously provided wherein a reporter gene is operably linked to the genetically-engineered promoter of the invention. Suitable reporter genes include but are not limited to luciferase, β-galactosidase, dihydrofolate reductase, thymidine kinase, chloramphenicol acetyl transferase, green fluorescent protein, hygromycin resistance, P-glycoprotein, neomycin resistance or any other gene whose expression provides a suitable means for phenotypic selection. Reporter-gene encoding recombinant expression constructs are useful, inter alia, for optimizing expression regulation by small molecule regulators.

The terms “expression construct” and “recombinant expression construct” will be understood to describe genetically-engineered nucleic acid sequences encoding at a minimum an origin of replication, a selectable marker and a gene or polypeptide-encoding nucleic acid of interest to be expressed in a recipient host cell.

As used herein the term “operably linked” is intended to describe covalent linkage between nucleic acids wherein the quality, position and proximity of the linkage ensures coupled replication and is sufficient and appropriate to be recognized by regulatory proteins and other trans-acting transcription factors and other cellular factors whereby polypeptide-encoding nucleic acid is efficiently expressed under appropriate conditions.

As used herein the term “promoter” is intended to encompass any nucleic acid that mediates expression of a gene to which it is operably linked in a cell, most preferably a mammalian cell. Expression via a promoter of the invention is typically by transcription of the gene sequence from an initiation site adjacent to the promoter, most preferably a site positioned between the promoter sequence and the protein-coding gene sequence, Representative and exemplary promoters comprise sequences such as AT-rich sequences termed “TATA” boxes, and additional sequences comprising the sequence “CAAT” that are recognized as mediating the interaction of the nucleic acid of the promoter with protein factors such as RNA polymerase. Preferably, the promoter is a mammalian MLCK promoter, and more preferably a promoter comprising one or a plurality of mutations that increase transcription from the promoter in the presence of a transcription factor produced in a cell by stimulation of a signaling pathway comprising a Ras oncogene.

The term “regulatable promoter” is intended to encompass DNA sequences that mediate transcription of a nucleic acid molecule in a cell. In addition to the features and properties possessed by promoters generally, regulatable promoters are distinguished from promoters that are not regulatable in that regulatable promoters are operatively linked to “cis-acting transcription control elements” that will be understood to be nucleic acid sequences that regulate or control transcription of a polypeptide-encoding nucleic acid. As used herein, the term “cis-acting transcription control element” is particularly directed to nucleic acid sequences that make said regulatable promoter “inducible,” as that term is defined herein below. Said regulatable promoters of the invention comprising said cis-acting transcription control elements are operatively-linked to polypeptide-encoding nucleic acids and control transcription thereof in a cell, most preferably a mammalian cell, into which a recombinant expression construct of the invention has been introduced. Most preferably the transcription control of the regulatable promoters of the invention shows increased transcription from the promoter in the presence of a transcription factor produced in the cell by stimulation of a signaling pathway comprising a Ras oncogene.

The term “inducible” will be understood to mean that activation of transcriptional activity of a regulatable promoter comprising a cis-acting transcriptional control element is initiated or increased by a stimulus. Preferably, the inducing stimulus is an alteration in the cell, including but not limited to the presence of a transcription factor produced in the cell by stimulation of a signaling pathway comprising a Ras oncogene.

The invention also provides recombinant cells comprising the recombinant expression constructs of the invention, wherein regulated expression of a gene encoded by the construct can be achieved. In certain preferred embodiments, the cells are cell lines, either established cell lines such as COS-7 or HEK29.3 cells as are available, for example, from the American Type Culture Collection (Manassas, Va.) or primary cells and cell lines, such as primary cultures of fibroblasts, hematopoietic cells, and germ cells. In these embodiments, the recombinant expression constructs of the invention are introduced into the cells using methods well-known in the art, including but not limited to electroporation, transfection using calcium phosphate co-precipitate or lipid-mediated transfection, or viral infection. The choice of the method used to introduce the recombinant expression construct of the invention into a particular cell or cell line is within the skill of the ordinarily skilled worker and can be adapted to the cell or cell line without changing the character or effectiveness of the invention. In certain other embodiments, the cells comprise a tissue, either in vivo or ex vivo, and the recombinant expression constructs can be introduced into cells in the tissue either specifically (for example, by targeting certain cell types for infection or by targeting with lipids or liposomes with or without cell type-specific molecules embedded therein), or non-specifically, most directly by simple injection as disclosed in U.S. Pat. No. 5,580,859 (incorporated by reference herein). Alternatively, infection using recombinant adenovirus (as disclosed, for example, in U.S. Pat. No. 5,880,102, incorporated by reference herein), recombinant adeno-associated virus (as disclosed, for example, in U.S. Pat. No. 5,622,856, incorporated by reference herein), or recombinant retroviral vectors (as disclosed, for example, in U.S. Pat. No. 5,952,225, incorporated by reference herein) can be used. Alternative methods include electroporation (as disclosed, for example, in U.S. Pat. No. 5,983,131, incorporated by reference herein) and lipid or liposome-mediated introduction of exogenous DNA (as disclosed, for example, in U.S. Pat. No. 5,703,055, incorporated by reference herein).

The invention further provides methods for identifying a compound that induces gene expression from a recombinant expression construct provided herein, comprising the steps of: (a) contacting a recombinant mammalian cell comprising said recombinant expression construct with the compound; (b) comparing gene expression from the recombinant expression construct in the presence and absence of the compound; and (c) identifying a compound that induced expression from the recombinant expression construct when gene expression is higher in the presence than in the absence of the compound. In certain embodiments, the compounds identified in the methods of the invention can be used for treating hypertension and for reducing ceil proliferation as described herein.

In addition, the invention provides methods for identifying a compound that decreases angiotensin-induced gene expression from a recombinant expression construct provided herein, comprising the steps of: (a) contacting a recombinant mammalian cell comprising said recombinant expression construct with the compound in the presence and absence of angiotensin; (b) comparing gene expression from the recombinant expression construct in the presence and absence of the compound; and (c) identifying a compound that decreases angiotensin-induced gene expression from a recombinant expression construct when gene expression is lower in the presence than in the absence of the compound. In certain embodiments, the compounds identified in the methods of the invention can be used for treating hypertension and for reducing cell proliferation as described herein.

The following Examples illustrate certain aspects of the above-described method and advantageous results. The following examples are shown by way of illustration and not by way of limitation.

### EXAMPLE 1

### Genetic Mutation Related to Hypertension in Rat Animal Model

MLCK is a key regulator of smooth muscle contraction and cell proliferation. Therefore, mutations in the MLCK promoter could increase MLCK expression, MLC-P and the contraction and proliferation of smooth muscle cells in hypertension. To investigate this possibility, the structure of the rat MLCK gene (EnsEMBL transcript ID-ENSRNOT00000036465) was analyzed and compared to cDNA sequences from the mouse (GenBank accession no. AY2377.27) and chicken (GenBank accession no. M31048). This analysis showed that the translation start site (ATG) of the rat smooth muscle MLCK is in exon 15 (FIG. 1A). Since prior studies on the telokin promoter showed that the promoter was located in the preceding intron (Birukov et al, 1998, Id.), rapid amplification of 5′cDNA ends (5′RACE) was performed on total RNA isolated from rat aorta to identify the transcriptional start site of the MLCK RNA.

These experiments were performed as follows. Genomic DNA was extracted from the spleens of hypertensive (SHR), Sprague-Dawley (SD) and normotensive (WKY) rats using a DNeasy tissue kit according to the manufacture's instructions (Qiagen, Valencia, Calif.). PGR amplification was then carried out on genomic DNA using 5 μM of each primer (Forward, 5′-AAGCCTAGCCAGGTCTCCCAC-3′ SEQ ID NO: 13; Reverse, 5′-CTGCAATAACCAGTGAAGGAA-3′ SEQ ID NO: 14) (FIG. 1 A). PGR conditions were 1 min at 94 ° C., 1 min at 50 ° C. and 1 min at 72 ° C. for 35 cycles with 0.4 unit of Taq polymerase (Bioline, Valley Park, Mo.). The PCR products were cloned into a Topo vector (Invitrogen, Carlsbad, Calif.) and subjected to DNA sequencing using an AB1 Prism 3100 Genetic Analyzer (Applied Biosystems, Inc., Foster City., Calif.). 5′RACE (Rapid Amplification of 5′ Complementary DNA Ends) was performed on 5 μg of total RNA isolated from aortas of WKY rats using the GeneRacer Kit according to the manufacturers' instructions (Invirogen, Carlsbad, Calif.). The 5′ end of smooth muscle myosin light chain kinase mRNA was then amplified using a GeneRacer 5′ Nested primer and a gene specific primer: GTCCTTCAGGTCGTCTTCTGATACGG (SEQ ID NO: 2). The RT-PCR product obtained in this manner was then cloned into the pCR2.1-Topo vector and sequenced.

Sequencing the cloned PCR product revealed that the transcription initiation site resides in the intron between exons 14 and 15 (intron 14-15) of the MLCK gene, which is 363 bp upstream of the initiation codon in exon 15 (shown in FIGS. 1A & 1B). The results obtained from isolated intron 14-15 of the MLCK gene from genomic DNA from SHR and WKY rats were analyzed for cis-acting transcription elements using the Transcription Element Search System (University of Pennsylvania). This analysis showed that intron 14-15 contains a TATA box and multiple Ras responsive elements, including a CArG box that binds serum response factor (SRF) (FIG. 1B). The TATA and CArG boxes are located 89 and 168 bp upstream of the transcription start site, respectively.

Comparison of the nucleotide sequences of intron 14-15 from normotensive (WKY) and hypertensive (SHR) rats showed that the SHR sequence contains a 12 bp insertion consisting of six pairs of CT repeats that is not found in normotensive WKY or Sprague-Dawley rats (FIGS. 1B and 1C). The insertion is located 11 bp upstream of a CArG box (FIG. 1B). Intron 14-15 from WKY rats also contains 3 different single nucleotide polymorphisms compared to intron 14-15 from SHR and other rat strains (FIG. 1B). Further analysis showed that intron 14-15 from stroke-prone SHR (SHRSP), a closely related genetic strain of SHR that have an even higher blood pressure than SHR and a high incidence of strokes (Yamori, 1984, Id.), also contain the 12 bp insertion (FIG. 1C). These results suggested that genetic mutation, including the SNPs and particularly the 12 bp insertion, could be a common aspect of hypertension in the SHR and SHR-SP hypertension model rats.

### EXAMPLE 2

### MLCK Promoter Activity in Normotensive and Hypertensive Rats

The presence of a TATA box suggested that intron 14-15 contains the smooth muscle MLCK promoter that had many different binding sites for transcription factors. Therefore, electiomobility shift assays (EMSA) were performed to determine if TATA binding proteins (TBP) binds to the TATA box in intron 14-15, a key step in defining the promoter.

Electromobility Gel Shift Assay: Intron 14-15 or the oligonucleotides representing defined regions of the SHR or WKY MLCK promoters (shown in FIG. 2B) were 5′-end-labeled with T4 polynucleotide kinase (Invirogen) and [γ-³²P]-ATP. The binding reaction was carried out at room temperature for 30 min in a total volume of 10 μl containing ˜10,000 cpm (1-5 ng) of the radiolabeled DNA, 2 μg of the nuclear extract and 1 μg of poly (dI-dC). For antibody supershift assays, 1.2 μg of antibody to SRF (Santa Cruz Biotechnology, Santa Cruz, Calif.) was added and the reaction mixtures were incubated for an additional hour at 4° C. with gentle shaking. The reaction mixtures were then loaded on 4% native polyacrylamide gels and electrophoresis carried out at 350 volts for 30 min. Radioactive bands were visualized by autoradiography.

These assays showed that TBP interacts with intron 14-35 from SHR in a concentration dependent manner (FIG. 2A), thereby confirming that intron 14-15 contains at least part of the promoter region of smooth muscle MLCK.

Since nearly all contractile proteins contain at least one CArG box in their promoters, and serum response factor (SRF) Is considered to be an important regulator of contractile protein expression (reviewed in Miano, 2003, J. Mol. Cell. Cardiol., 35: 559-708), SRF binding to the CArG box in the intron 14-15 was examined. An additional reason for examining these interactions is that if SRF plays an important role in regulating smooth muscle MLCK expression then the 12 bp insertion could affect SRF binding to the CArG box in SHR. To analyze these interactions specifically, two different oligonucleotides that represent defined areas of intron 14-15 from SHR and WKY rats were synthesized. These oligonucleotides contain the CT repeats and the CArG box (FIG. 2B, box) and the only difference between them is the 12 bp CT repeat found in SHR. An EMSA with nuclear extracts from cells expressing SRF and ³²P-labeled WKY or SHR oligonucleotides showed that the formation of SRF-DNA complexes increased with increasing concentration of nuclear extracts and that the signal was always more intense when incubated with SHR oligonucleotides than with WKY oligonucleotides (FIG. 2B).

Chromatin immunoprecipitation (ChIP) assays were used to directly determine whether SRF binds to the CArG box found in intron 14-15 of the MLCK gene. ChIP assays were performed on VSMC isolated from aortas of SHR or WKY rats essentially as previously described (Hofmann et al., 2004, Id). The β-globin promoter, which is silent in VSMC (Manabe & Owens, 2001, Id,% was used as a negative control. These assays were performed as follows.

Chromatin Immunoprecipitation Assays: ChIP assays were performed as described by Hofmann et al (2004, Nature Cell Biol. 6: 1094-1101), with some modifications. Briefly, VSMC from WKY rats were fixed directly with formaldehyde. Cross-linked chromatin was immunoprecipitated with antibodies to SRF (Upstate, Lake Placid, N.Y.) or RNA polymerase II (Hofmann et al., 2004, Id.). The precipitated chromatin DNA was then purified and subjected to PCR analysis. β-globin promoter-specific primers were designed as described (Manabe Sc Owens, 2001, J. Clin Invest. 107: 823-834) (Forward, 5′-CAGCGTTTTCTTCAGAGGGAGTACCCAGAG-3′ SEQ ID NO: 15; Reverse, 5′-TCAGAAGCAAATGTGAGGAGCGACTGATCC-3′ SEQ ID NO: 16); MLCK promoter-specific primers were 5′-TCAGGAACCGGGTTGGCGAATGCA-3′ (SEQ ID NO: 3) and 5′-TGCATTCGCCAACCCGGTTCCTGA-3′ (SEQ ID NO: 4).

After visualizing PCR products on ethidium bromide-stained agarose gels, fragment band densities were measured using a densitometer. The relative enrichment of intron 14-15 was determined by calculating the ratio of intron 14-15 present in the immunoprecipitates compared with intron 14-15 in the input chromatin.

These results are shown in FIGS. 2C and 2D. Antibodies to SRF or RNA polymerase II failed to precipitate DNA containing the β-globin promoter (FIG. 2C). However, these antibodies specifically enriched intron 14-15 of the MLCK gene. Comparing the binding of SRF to intron 14-15 from SHR and WKY rats, following normalization to the input chromatin, showed that SRF binding is greater to intron 34-15 from SHR compared to WKY rats (FIG. 2D). These data demonstrate a direct, in vivo interaction of SRF with intron 14-15 of the MLCK gene and, along with data from the EMSA, strongly support the idea that the 12 bp insertion in the SHR intron increases SRF binding to the CArG box, in vitro and in vivo.

### EXAMPLE 3

### Increased Responsiveness of the SHR MLCK Promoter to SRF

The effect of the 12 bp insertion on promoter activity was further investigated using luciferase reporter gene assays. Intron 14-15 from SHR, WKY rats and Sprague-Dawley rats, a separate strain of normotensive rats that does not have the 12 bp sequences (FIG. 1C), were cloned into luciferase vectors (FIG. 3A, diagram) and used to transfect Cos-7 cells; these experiments were performed as follows.

Reporter Activity Assays. Introns 14-15 from SHR and WKY rats were cloned into a pGL3-Basic Firefly luciferase vector (Promega, Madison, Wis.). To create a mutated construct, the 12 bp sequence found in SHR was then inserted into the intron 14-15 from WKY using QuikChange XL Site-Directed Mutagensis Kit (Stratagene, La Jolla, Calif.). The integrity of the constructs was confirmed by DNA sequencing. These constructs and a CMV-Renillar luciferase vector (Promega) were co-transfected into COS-7 cells. Firefly and Renillar luciferase activities were measured using a dual luciferase assay system (Promega) and the ratio of Firefly:Reniifar luciferase activities were calculated to correct for differences in transfection efficiency. When the intron 14-15/Firefly luciferase was co-transfected with SRF (Chen & Schwartz, 1996, Mol. Cell Biol. 16: 6372-6384), N17Ras or N19Rho expression vectors, the Renillar vector was not used to avoid complexity of triple transfection. In this case, the luciferase activity was normalized to protein concentration in the cell extract.

Luciferase assays demonstrated that intron 14-15 from all 3 rat strains have strong basal promoter activities (5.1×10⁶, 4.0×10⁶ and 7.6×10⁶ RLU/mg for WKY, Sprague-Dawley and SHR, respectively). Co-transfection with SRF increased promoter activities in a concentration dependent manner (FIG. 3A). Importantly, SRF increased the promoter activity of intron 14-15 from SHR more than WKY or Sprague-Dawley rats.

The data described in FIG. 3A suggest that the 12 bp insertion is responsible for the increased promoter activity in SHR. Because the 12 bp insertion is the major difference between introns 14-15 in SHR and WKY rats, adding the 12 bp insertion was expected to stimulate the activity of the WKY promoter. Therefore, site directed mutagenesis was used to add the 12 bp sequence to intron 14-15 of WKY rats (FIG. 3B, diagram). Luciferase assays showed that cells co-transfected with SRF and either the mutated WKY promoter or the SHR promoter had similar levels of activity (FIG. 3B). These data demonstrated that the 12 bp insertion was sufficient to increase MLCK promoter activity in response to SRF,

### EXAMPLE 4

### Regulation of MLCK Expression and MLC-P by the Ras-MEK-ERK Cascade In Vitro

In view of the results obtained in Example 3 above, the effect of modulating SRF expression or activity on MLCK expression was assayed. To inhibit SRF activity, an adenovirus (AdSRF-S) lacking exons 4 and 5 was used to express a truncated, dominant negative form of SRF in cells (Davis et al., 2002, Id). Control cells were infected with a virus expressing GFP (AdGFP) at the same multiplicity of infection. In these experiments, VSMC were explanted from aortas of 4 to 7 weeks old SHR or WKY rats as previously described (Ross, 1971, J. Cell. Biol. 50: 172-186) and cultured in Dulbeccos's modified Eagles's medium (DMEM) with 10% fetal bovine serum (FBS). VSMC from WKY rats grown in 6-well plates (90-100% confluent) were infected for 2 hours with either an adenovirus that express a short form of SRF (AdSRF-S) (Davis et al., 2002, Id) or a control virus that expresses GFP (AdGFP). The viruses were washed out and the cells were grown as described above for 48 hours. VSMC were harvested and cell lysates were subjected to western blot analyses.

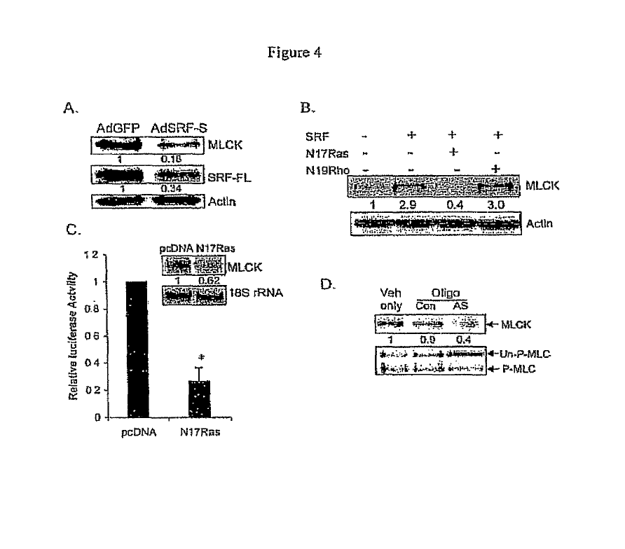

The results of these experiments showed significant decreases in full length SRF and smooth muscle MLCK in VSMC infected with the AdSRF-S virus compared to cells infected with the control AdGFP virus (shown FIG. 4A).

In view of the known ability of the Ras oncogene to regulate c-fos expression via activation of SRF (Wang & Olson. 2004, Id), and that Ras signaling also plays an important role in hypertension and SRF stimulates MLCK promoter activity (Chen et al., 2004, Id; Chien & Hoshijima, 2002, Id,), Ras regulation of MLCK expression through SRF was investigated. VSMC from WKY rats were co-transfected with SRF and control plasmids or plasmids expressing dominant-negative Ras (N17Ras), as well as with SRF and dominant-negative Rho (N19Rho) (because Rho is also reported to affect transcription via SRF1 Miralles et al., 2003, Cell 113: 329-342). VSMC were extracted 2 days after co-transfection and western blot analyses were performed using antibodies to MLCK. SRF increased MLCK expression (FIG. 4B, lane 2) and co-transfection of dominant negative Rho did not affect the SRF-induced increase in MLCK expression (FIG. 4B, lane 4). In contrast, the increase in MLCK expression induced by SRF was blocked by co-transfection with dominant-negative Ras (FIG. 4B, lane 3). These data demonstrated that SRF-induced MLCK gene expression in VSMC was regulated by Ras and not by Rho.

The relationship between Ras signaling and the regulation of MLCK expression at the transcriptional level was next investigated. VSMC from SHR were transfected with plasmids expressing pcDNA or N17Ras as described above, and reporter gene activity of the MLCK promoter from SHR was measured. Total RNA was also isolated from these cells and Northern blot analyses were performed using a ³²P-labeled MLCK cDNA fragment. In these assays, total RNA was extracted from aortas of 14 week old SHR and WKY rats using a RNeasy Fibrous Tissue Mini Kit (Qiagen Sciences, Valencia, Calif.). Purified RNA (2 μg) was separated on 1% agarose-formaldehyde denaturing gels and transferred to nylon membranes. A 1790 bp fragment of the MLCK cDNA (amino acids 426 to 1022 based on rabbit smooth muscle MLCK cDNA) labeled with [α³²P]dCTP was used to probe the blots. The blots were washed 4× with 150 mM NaCl, 20 mM sodium citrate, pH 7 (SSC) containing 0.1% sodium dodecyl sulfate (SDS) and 2 times with 0.5× SSC containing 0.1% SDS. The blots were then subjected to autoradiography and the amount of mRNA in each band was quantified densitometrically.

The results of these assays showed that dominant negative Ras significantly decreased the activity of intron 14-15 from SHR (FIG. 4C). Dominant negative Ras decreased MLCK mRNA expression in VSMC from SHR (FIG. 4C, inset). To investigate downstream events of Ras signaling, VSMC from SHR were treated with antisense oligonucleotides to ERK, a known member of the Ras signaling pathway. In these experiments, VSMC were transfected with control oligonucleotides (scrambled sequence) or antisense oligonucleotides (Robinson et al., 1996, Biochem, J. 320: 123-127) to ERK (5 μM each, Calbiochem, San Diego, Calif.) using lipofectamine. Antisense oligonucleotides to Erk inhibited MLCK expression and MLC-P in VSMC from SHR (FIG. 4D, lane 3) while scrambled oligonucleotides (control) had no effect (FIG. 4D, lane 2).

These results provided further confirmation that the MLCK intron 14-15 promoter, and thus MLCK gene expression, was responsive to the Ras signaling cascade.

### EXAMPLE 5

### Regulation of MLCK Expression and MLC-P by the Ras-MEK-ERK Cascade In vivo

In view of these results, blood vessels from SHR and WKY rats were analyzed to determine if components of the Ras and MLCK signaling pathways are altered in SHR. Cells or pieces of blood vessels were extracted in buffer containing 9 M urea, 10 mM DTT and 20 mM Tris, pH 8.0. Protein concentrations were measured using the Bradford Protein Assay (BioRad, Richmond, Calif.) and equivalent amounts of protein were subjected to SDS-PAGE or urea-glycerol PAGE, Proteins separated by SDS-PAGE were transferred to nitrocellulose and probed with an antibody to MLCK (de Lanerolle et al., 1981, Proc. Natl. Acad. Sci. 78: 4738-4742), the broad specificity C4 antibody to actin (Lessard, 1988, Cell. Motil. Cytoskeleton. 10: 349-362), antibodies to Rho A or phosphorylated MPasel (Santa Cruz Biotechnology, Santa Cruz, Calif.) or an antibody to phosphorylated ERK½ (Cell Signaling Technology, Beverly, Mass.). Urea-glycerol PAGE was used to separate the unphosphorylated and phosphorylated forms of MLC₂₀, which was then transferred to nitrocellulose and probed with an antibody to MLC₂₀, wherein the stoichiometry of phosphorylation (mol PO₄/mol MLC₂₀) was calculated as previously described (Obara et al., 1989, Pflugers. Arch. 414: 134-138). Ail immunoreactive bands were visualized using enhanced chemiluminescence (Amersham Pharmacia Biotech, Piscataway, N.J.) and quantified densitometrically.

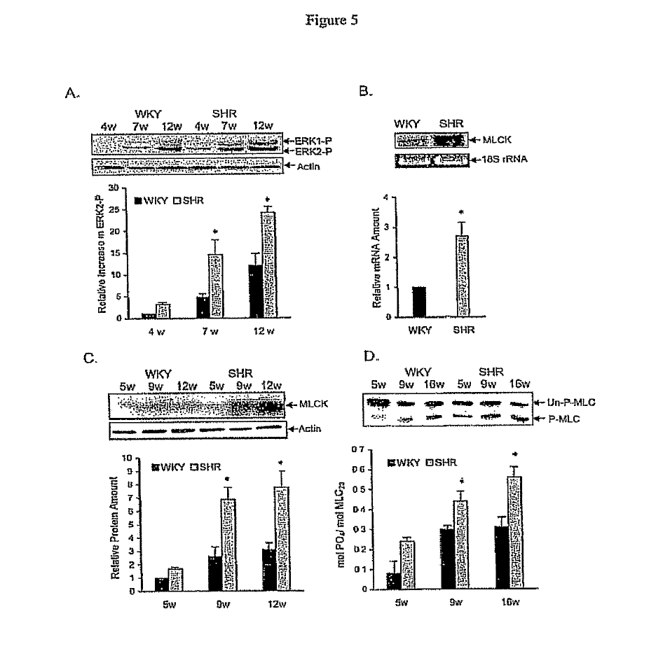

Western blot analyses showed that the active, phosphorylated form of ERK (ERK-P) increased in blood vessels as rats age (FIG. 5A) and that the level of ERK-P w as significantly higher in SHR compared to age-matched WKY rats (FIG. 5A). Northern blot analyses, performed as described above, showed a 2.7±0.45 fold increase of MLCK mRNA in blood vessels from adult SHR compared to age-matched WKY rats (FIG. 5B). The increase in mRNA levels was accompanied by increases in MLCK protein and MLCK protein levels were always higher in SHR compared to age-matched WKY rats (FIG. 5C). MLC-P in blood vessels also increased with age and MLC-P was always greater in SHR compared to age-matched WKY rats (FIG. 5D). It is worth emphasizing that blood pressure increases with age in SHR (Yamori, 1984, Id.) and that the increases in ERK-P, MLCK protein levels and MLC-P temporally coincided with the increase in blood pressure.

The importance of regulating MLCK expression by Ras signaling in vivo was further investigated by treating SHR with U0126 continuously for 3 weeks. U0126 is a specific inhibitor MEK (Favata et al., 1998J. Biol. Chem. 273: 18623-18632), an upstream regulator of ERK in the Ras signaling pathway (Chien & Hoshijima, 2002, Id.). Rats that were 8 weeks old at the start of treatment were studied because blood pressure increases and blood vessels become thicker between 8 and 12 weeks of age (Yamori, 1984, Id). In these experiments, Systolic blood pressure (SBP) was measured using tail-cuff sphygmomanometry (IITC Incorporated/Life Sciences Institute, Woodland Hills Calif.). Drugs were delivered via an osmotic pump (DURECT Corporation, Cupertino, Calif.) implanted subcutaneously in the shoulder. The pumps (having a 2 mL capacity at a delivery rate of 2.45 μL/hour) were filled with 13.5 mM U0126 (BIOMOL Research Laboratories, Plymouth Meeting, Pa.) dissolved in 50% DMSO or vehicle alone. Three weeks later, rats were sacrificed and aortas were removed. Some vessels were processed for histology, while other tissue was immediately placed in ice-cold acetone containing 10% trichloroacetic acid (TCA) and 10 mM dithiothreitol (DTT). Endothelial cells were removed by gently rubbing the inside of the vessels with a cell lifter and connective tissue was removed by dissection. The cleaned vessels were then frozen on dry ice and stored at −80° C. for biochemical analysis.

In SHR receiving vehicle (DMSO), the systolic blood pressure averaged 159.0±8.3 mmHg at the start of the experiment and increased to 208.9±4.7 mmHg by the end of the 3-week treatment period (FIG. 6A). In contrast, U0126 decreased systolic blood pressure to 150.4±4.1 mmHg.

ERK-P, MLCK expression and MLC-P were assayed in U0126 SHR as described above. The results of these experiments, shown in FIG. 6B, established that ERK and MLC phosphorylation and MLCK expression were decreased in blood vessels removed from U0126-treated SHR compared to control (FIG. 6B).

Blood vessels were also assessed histologically. Sections of the aorta immediately proximal to the superior mesenteric artery and the superior mesenteric artery immediately distal to the aorta were fixed in formalin and embedded in paraffin blocks. Cross-sections (5 microns thick) were cut, deparaffinized and stained with Gomort's Trichrome. Nuclei were visualized with Weigert's iron hematoxylin. The thickness of the medial layer was quantified using NIH ImageJ 1.32 software (National Institutes of Health, Bethesda, Mass.).

Histological analysis of the blood vessels revealed a thinner medial smooth muscle layer in both aortas and mesenteric arteries removed from rats receiving U0126 compared to controls (FIG. 6C). The total medial area of aortas measured by NIH ImageJ software was 117.8±3.69 (relative units) for SHR that received vehicle and 66.4±2.79 (P<0.05 vs. vehicle) for SHR that received U0126. The total medial area of mesenteric arteries was 26.88 for SHR that received vehicle and 16.0 for SHR that received U0126 (the mean from two animals). These data demonstrated that inhibiting MEK decreased ERK activation, MLCK expression, MLC-P and VSMC proliferation and inhibited the development of hypertension in SHR. Moreover, they established the importance of this pathway in vivo.

Another experiment was conducted to determine if inhibiting Ras signaling could decrease blood pressure in SHR. U0126, delivered for 3 weeks using an osmotic pump, significantly decreased blood pressure in SHR that were 16 week old at the start of the experiment. The mean systolic blood pressure was 181.4±0.89 mmHg in SHR receiving U0126 compared to 221.6±4.2 mmHg in SHR receiving vehicle at the end of the 3-week treatment period (FIG. 7). U0126 resulted in a transient decrease in SBP in the normotensive WKY rats that returned to baseline within 2 weeks of treatment (FIG. 7).

In addition, MLCK activity was inhibited with ML-7, a specific inhibitor of MLCK (Fazal et al, 2005, Mol Cell Biol. 25:6259-62661, to determine is directly inhibiting MLCK could decrease blood pressure. An osmotic pump was used to deliver ML-7 or vehicle (DMSO) continuously for 3 weeks to SHR that were 16 weeks old at the start of the experiment (FIG. 8). ML-7 resulted in a significant decrease in SBP (165.0±3.9 mmHg) compared to SBP in SHR receiving DMSO (221.6±6.8 mmHg).

### EXAMPLE 6

### ML-7 Induces Apoptosis in Mammary and Prostate Cancer Cells

ML-7 induces apoptosis in smooth muscle cells (Fazal el al, 2005, Mol Cell Biol 25:6259-66), To determine if ML-7 has a similar effect on cancer cells, Mm5MT mouse mammary cancer cells (American Type Culture Collection, Manassas, Va.) and MLL rat prostate cancer cells (Mat-Ly-Lu subline of Dunning R-3327 prostate adenocarcinoma) were treated with varying concentrations of ML-7 for 16 hours. The Mm5MT cell line was maintained in DMEM medium supplemented with 10% fetal bovine serum (FBS) and 100 U/ml penicillin, 100 μg/ml streptomycin. The MLL cells were maintained in RPMI1640 medium supplemented with 10% FBS, 250 nM dexamethasone, 100 U/ml penicillin and 100 μg/ml streptomycin. Mm5MT or MLL cells (200,000 cells per well) were seeded in 6-well dishes one day before drug treatment and cultured as described above. The cells were collected and apoptosis was quantified as follows.

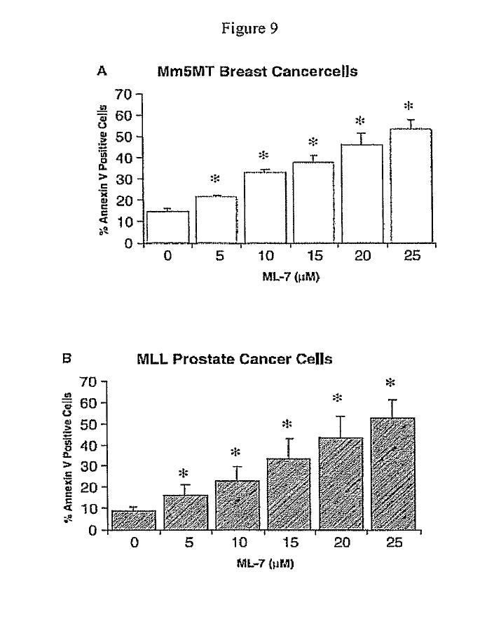

On the day of the experiment, media was changed to DMEM supplemented with 0.5% FBS and the cells were treated with drugs as defined by the specific experimental protocol. ML-7 (Biomol, Plymouth Meeting, Pa.) was incubated with cells for 16 hours. The cells were then treated with trypsin, washed twice with cold PBS and re-suspended in 100 μl of buffer containing 10 mM Hepes, pFI 7.4, 140 mM NaCl and 2.5 mM CaCl2 (binding buffer). Then, 5 fit of FITC-conjugated annexin V (Pharmingen, San Diego, Calif.) and 10 μl of propidium iodide (PI) (50 μh/ml) were added and cells were incubated in the dark at room temperature for 15 min. Next, 400 μl of binding buffer was added per sample and the cells were analyzed cytoflourometrically using a Coulter Epics Elite ESP flow cytometer (Ex: 488 nm, Em: 585 nm). At least 10,000 cells were counted per analysis and cells that stained positive for annexin V and PI were judged to be apoptotic. The results demonstrated that ML-7 induced a dose-dependent increase in apoptotic cells in both Mm5MT and MLL cells (FIG. 9).

### EXAMPLE 7

### ML-7 has a Chemopreventive Effect in an In Vitro Mammary Cancer Model

To determine the effects of inhibiting MLCK in mammary tumors, an in vitro mammary cancer model was used as follows. Mammary glands obtained from young Balb/c mice that are exposed to 7,12-dimethylbenz(a)anhracene (DMBA) for 24 hours in culture form precancerous lesions in 24 days (Mehta et al., 2000, J Natl Cancer Instit 92:418-23). In this experiment, 70 mammary glands from 35 Balb/c mice were divided into seven groups of 10 glands each and incubated in serum-free medium containing insulin, prolactin (5 μg/ml each), aldosterone and hydrocortisone (1 μg/ml each) for 10 days. DMBA (2 μg/ml) was included in the medium for 24 hours on day 3. The glands were incubated for an additional 14 days in the absence of hormones except insulin. This allows the regression of the normal mammary alveolar structures. The precancerous mammary alveolar lesions (MAL) acquire altered hormonal responsiveness do not regress under these conditions. Chemopreventive agents were included in the medium during the first 10 days. The glands were fixed in formalin and stained with alum carmine and evaluated for MAL. Percent inhibition was calculated by comparing the incidence in the control glands with the treated groups.

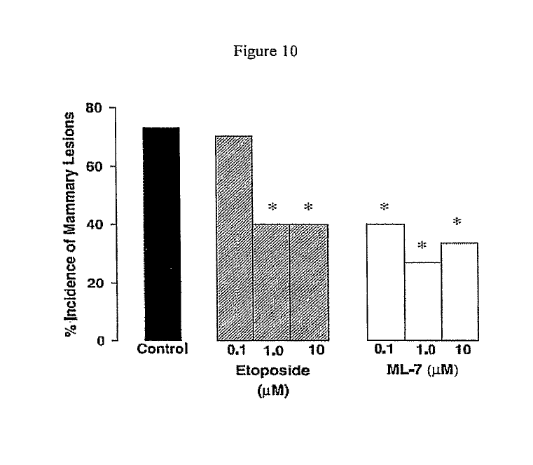

The effects of etoposide (Calbiochem, La Jolla, Calif.) and ML-7 at 0.1, 1.0 and 10.0 μM concentrations were examined on the development of MAL in organ culture. As shown in FIG. 10, etoposide inhibited the incidence of lesion formation at 1 and 10 μM concentration by 40-46% compared to control. Etoposide at 0.1 μM, however, did not affect MAL formation. ML-7 suppressed the development of MAL by 40% even at 0.1 μM and further reduced it to 58% of control at 1 μM. The differences observed between 0.1 and 10 μM ML-7 were not statistically different. However the inhibition of 58% at 1 μM compared to a 70% incidence in the control glands (7/10 glands positive) was significant.

### EXAMPLE 8

### ML-7 Stimulates the Ability of Etoposide to Induce Apoptosis in Mm5MT Cells

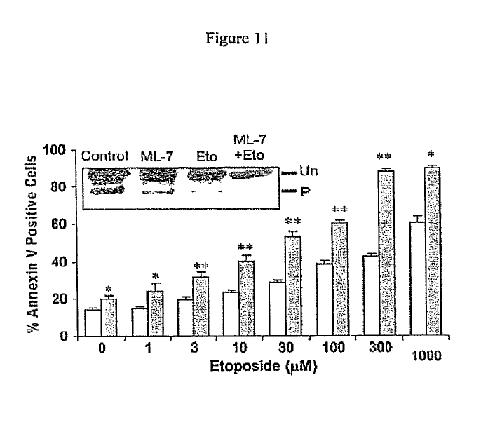

The combined effects of ML-7 and etoposide was examined in mouse Mm5MT. ML-7 was added to cells 2 hours before adding various concentrations of etoposide (1-1000 μM) and the cells were incubated with ML-7 and etoposide for an additional 16 hours. The cells were then treated with trypsin, washed twice with cold PBS and re-suspended in 100 μl of buffer containing 10 mM Hepes, pH 7.4, 140 mM NaCl and 2.5 mM CaCl2 (binding buffer). Then, 5 μl of FITC-conjugated annexin V (Pharmingen, San Diego, Calif.) and 10 μl of propidium iodide (PI) (50 μg/ml) were added and cells were incubated in the dark at room temperature for 15 min. Next, 400 μl of binding buffer was added per sample and the cells were analyzed cytoflourometrically using a Coulter Epics Elite ESP flow cytometer (Ex: 488 nm, Em: 585 nm). At least 10,000 cells were counted per analysis and cells that stained positive for annexin V and PI were judged to be apoptotic ML-7 (10 μM), by itself, significantly increased apoptosis (0 etoposide, FIG. 11). ML-7 also significantly increased the ability of etoposide to induce apoptosis (FIG. 11). A curve-fitting program (Cricket Graph) showed that the concentrations of etoposide required for inducing apoptosis in 50% of the cells was 25.4 μM plus ML-7; and 572 μM minus ML-7.

MLC-P expression was measured in the cells using urea/glycerol gel-immunoblotting as follows. Cells treated with ML-7 (10 or 5 lM, respectively) or etoposide (30 lM) or combination of two at indicated concentrations for 16 hours were fixed in 10% trichloroacetic acid (TCA) containing 10 mM dithiothreitol (DTT), Cell pellets were washed four times with acetone and protein was extracted by dissolving in buffer containing 9 M urea, 10 mM DTT and 20 mM Tris, pH 8.0. The unphosphoryiated and phosphorylated forms of MLC20 were separated using urea/glycerol PAGE, transferred to nitrocellulose and probed with an affinity purified antibody to MLC20. This antibody recognizes the unphosphoryiated and phosphorylated forms of MLC20. Immunoreactive bands were visualized using enhanced chemiluminescence (ECL) detection reagents (Amersham Pharmacia Biotech, Piscataway, N.J.) (Fazal et al., 2005, Mol Cell Biol 25:6259-66). The results showed that both 10 μM ML-7 and 30 μM etoposide decreased MLC-P in Mm5MT cells and that the combination of the two drugs almost completely eliminated MLC-P.

### EXAMPLE 9

### ML-7 has an Additive Tumoricidal Effect with Etoposide on Mammary Cancer in Mice

To investigate the anticancer activity of ML-7 in vivo, Mm5MT cells were injected into the right flanks of female mice as follows. Mm5MT cells grown in culture were harvested immediately before injection into syngeneic MMTV-C3H/HeM mice. Cells were washed to remove serum and 10⁶ cells were resuspended in 100 μl of serum-free DMEM. Healthy, MMTV-free female mice (14-20 weeks old) were anesthetized with ether and 10⁶ cells were injected subcutaneously into the right flank. The mice were randomly divided into four groups of five mice, each, and treated with vehicle, ML-7, etoposide or ML-7 plus etoposide. Drug administration was started 1 week after the cells were injected. To deliver ML-7, a small horizontal incision was made in the interscapular area and a 200 μl osmotic pump (Alzet, Cupertino, Calif.) filled with either 27 mM ML-7 in 50% DMSO or 50% DMSO (vehicle control) was implanted and the wound closed. These pumps have a release rate of 0.25 μl/h and released drug at this rate for 4 weeks. When given, 25 mg/kg etoposide was injected intraperitoneally on the first 3 days of every week for 4 weeks (days 7-9, 14-16, 21-23 and 28-30) (Nakamura el al 2003, Cancer Sci 94:119-24). The mice were sacrificed with ether after 4 weeks of drug administration and tumors were removed, weighed and processed for analysis.

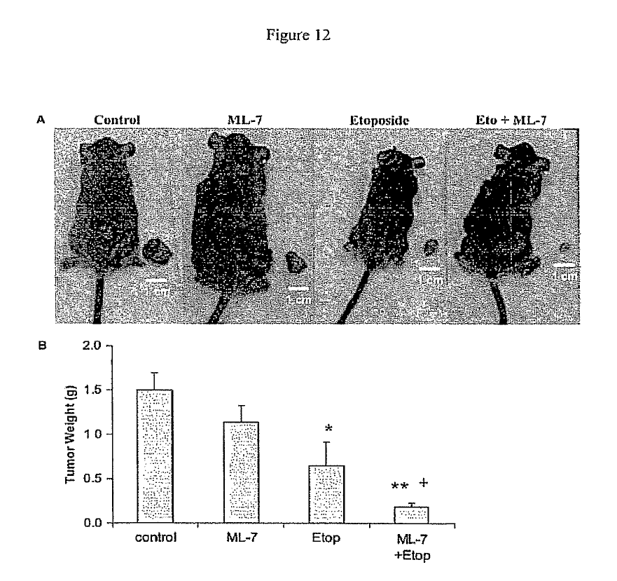

Physical examination of the mice showed that the mice treated with vehicle, ML-7 or etoposide alone tolerated these drugs without visible signs of discomfort (e.g., animals receiving ML-7 were active, there was no obvious loss of appetite, their breathing was not labored, they had no rectal bleeding and they put on weight). ML-7 and etoposide both decreased tumor growth, but only the etoposide effect was statistically significant (P<0.05) compared to mice receiving vehicle. Importantly, the combination of ML-7 and etoposide dramatically reduced tumor growth compared to mice receiving vehicle (88.5% inhibition of tumor growth, P<0.001) and to mice receiving etoposide alone (P<0.05) (FIG. 12).

Histological analysis of the tumors was conducted as follows. The excised tumours were gently patted dry and weighed using a Mettler digital balance. Tumors were then sectioned into 2 mm slices, fixed in 10% neutral buffered formalin, routinely processed and embedded in paraffin. Five micron sections demonstrating the entire surface were stained by hematoxylin and eosin and examined by a surgical pathologist blinded to the experimental conditions. Photomicrographs documenting the entire section were collected and areas of necrotic and viable tumor were determined using the manual tracing tools within MetaMorph 6.2 (Universal Imaging Corporation, Downingtown, Pa.).

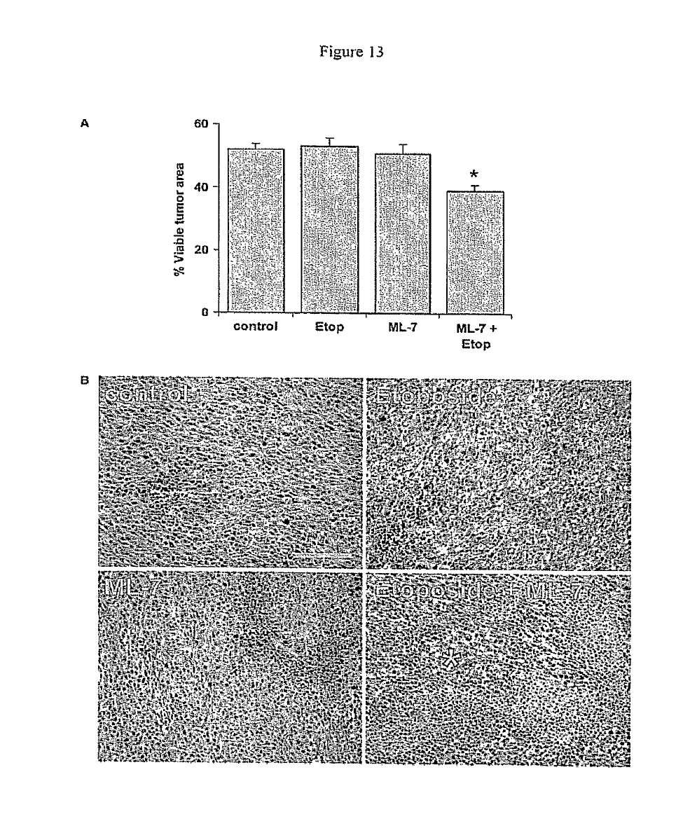

The analysis revealed significant necrosis within control, ML-7-treated, etoposide-treated, and ML-7 plus etoposide-treated mice, but significantly less viable tumor area in mice treated with etoposide and ML-7 (FIG. 13A). It was apparent on further examination that the distribution of necrosis in control, etoposide treated and ML-7-treated mice was predominantly confined to the center of the tumor. This pattern is typically seen in rapidly growing tumors. Away from these central areas, small foci of apoptosis were apparent, but adjacent tumor was viable, with intact cell adhesions, as evident by tumor cell cohesion, and readily identifiable mitotic figures (FIG. 13B). In contrast, necrosis in tumors of mice treated with ML-7 plus etoposide was distributed in a predominantly perivascular pattern (FIG. 13B). This pattern was clearly distinguishable from that seen in the other tumors and suggested that necrosis may have been induced by ML-7 and etoposide. Individual cells within these areas of necrosis were characterized by dense eosinophilic cytoplasm and shrunken fragmented nuclei, a morphology typical of apoptosis. Cells adjacent to these areas, that were not frankly necrotic, generally showed early signs of ceil death, including dis-cohesion, vacuolization and absence of mitoses. Thus, the in vivo synergy between ML-7 and etoposide causes a pattern of tumor necrosis consistent with tire enhanced apoptosis observed in vitro.

### EXAMPLE 10

### ML-7 Induces Apoptosis in Prostate Cancer Cells and has Tumouricidal Effects on Rat Prostate Cancer

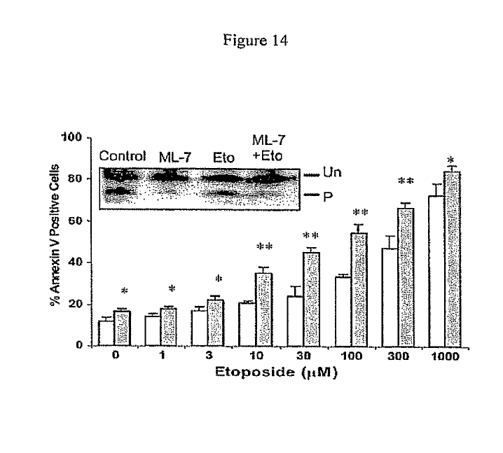

To determine if ML-7 stimulates the ability of etoposide to induce apoptosis and retard tumor growth widely, the effects of ML-7 and etoposide on MLL prostate cancer cells were determined. In this case, pre-treated MLL cells were grown in culture with 5 μM ML-7 before adding varying concentrations of etoposide from 1 to 1000 μlM. ML-7 significantly increased the apoptotic effect of etoposide when compared with cells treated with etoposide alone, and decreased the concentration required for inducing apoptosis in 50% of the cells from 376 μM (no ML-7) to 68 μM (with ML-7, FIG. 14). Urea/glycerol gel-immunoblotting showed that 5 μM ML-7 decreased MLC-P and that 30 μM etoposide resulted in a smaller decrease in MLC-P in MLL cells. When used together, MLC-P was decreased to a level comparable to ML-7 alone (FIG. 14, inset).

To test the anticancer effect of ML-7 in a rat prostate cancer model as follows. MLL cells grown in culture as described above were harvested and washed in serum-free Hank's buffer. The cells were suspended in 500 μl serum-free Hank's and 10⁶ cells were injected subcutaneously into the right flank of 12-week old male Copenhagen rats anesthetized with ether. The cells were allowed to grow and drug treatment was started 5 days after inoculation when the rats had developed palpable tumors. The rats were randomly divided into four groups, five in each group. The rats received injections of ML-7 or vehicle via the jugular vein every 4 days for 2 weeks. ML-7 was used at the dose of 35 mg/kg. Etoposide was injected intraperitoneal injection (IP) at the maximum tolerant dose of 50 mg/m² daily (Muenchen et al., 2000, Anticancer Res 20:735-40), The rats were sacrificed with ether 14 days after the start of drug treatment. The tumors were removed, weighed and processed as described above.

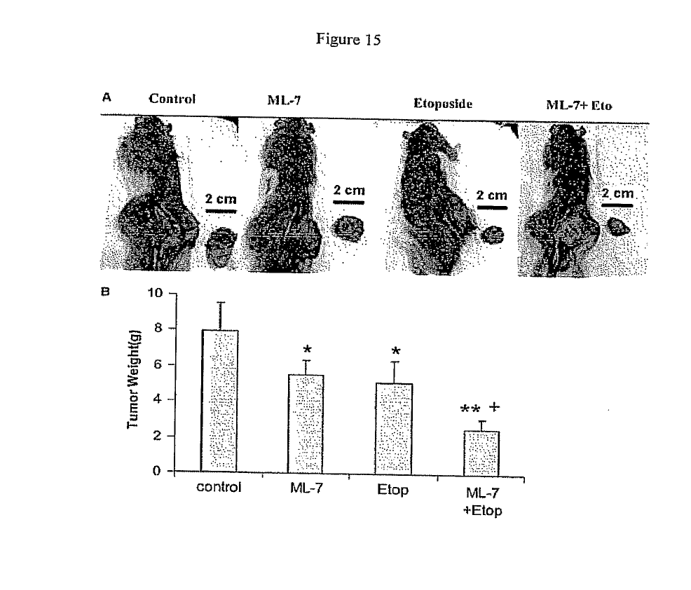

As with the mice, the rats appeared to tolerate the individual drugs or vehicle without obvious discomfort. Rats receiving both ML-7 and etoposide, however, appeared to be more lethargic and lost on average 15% of their initial body weight, ML-7 or etoposide atone significantly inhibited the prostate tumor growth and decreased tumor weight by 29.6% and 433%, respectively (P<0.05 vs. vehicle control). The combination ML-7 and etoposide further retarded tumor growth and decreased tumor weight by 79.1% compared to the vehicle control (P<0.001 vs. vehicle control) (FIG. 15).

Tumor sections were also examined for apotosis using TUNEL staining Sections were deparaffinized and rehydrated according to standard protocol. Tissue sections were permeabilized by placing slides in 10 mM citrate buffer (pH 6.0) and applying 350W microwave irradiation for 5 min. Tissue sections were then stained with TMR Red-labelled terminal deoxynucleotidyl transferase dUTP nick-end labeling (TUNEL) enzyme reagent using the In Situ Cell Death Detection kit (Roche Molecular Biochemicals, Indianapolis, Ind.) as described by the manufacturer. Sections stained with the labeling solution without the terminal transferase were used as negative control. Tissue sections were finally mounted using Vectashield containing DAPI and examined using a Zeiss LSM 510 laser confocal microscope.

The TUNEL staining showed more apoptotic cells in sections from rats receiving ML-7 or etoposide compared to vehicle control. Importantly, the combination of ML-7 and etoposide further increased the number of apoptotic cells. Quantification of the TUNEL positive nuclei in 300 cells from randomly chosen fields in each group showed that 19.2%, 40,6%, 35.8% and 66.7% of the nuclei were TUNEL positive in control, ML-7-treated, etoposide-treated, and ML-7 plus etoposide-treated tumors, respectively.

It should be understood that the foregoing disclosure emphasizes certain specific embodiments of the invention and that all modifications or alternatives equivalent thereto are within the spirit and scope of the invention as set forth in the appended claims.

What is claimed is:

1. A recombinant expression construct comprising an inducible promoter, wherein the inducible promoter comprises a promoter from a mammalian myosin light chain kinase (MLCK) bearing one or a plurality of mutations that increase transcription from the promoter in the presence of a transcription factor produced in the cell after stimulation of a cellular signaling pathway comprising a Ras oncogene.

2. The recombinant expression construct of claim 1, wherein the promoter is an MLCK promoter from a rat expressing the SHR phenotype.

3. The recombinant expression construct of claim 2, comprising a 12 basepair insertion having the sequence CTCTCTCTCTCT (SEQ ID NO: 1) inserted proximal to a CArG site.

4. The recombinant expression construct according to claim 1, wherein gene expression from the construct is induced in a cell comprising said construct by contacting the cell with angiotensin.

5. A recombinant expression construct according to claim 1 wherein the inducible promoter is operably linked to a reporter gene.

6. The recombinant expression construct of claim 5 wherein the reporter gene is firefly luciferase, chloramphenicol acetyltransferase, beta-galactosidase, green fluorescent protein, or alkaline phosphatase.

7. A recombinant mammalian cell comprising the recombinant expression construct of claim 1.

8. A method for identifying a compound that induces gene expression from a recombinant expression construct according to claim 1, comprising the steps of:
a) contacting a recombinant mammalian cell comprising said recombinant expression construct with the compound;
b) comparing gene expression from the recombinant expression construct in the presence and absence of the compound; and
c) identifying a compound that induced expression from the recombinant expression construct when gene expression is higher in the presence than in the absence of the compound.

9. The method of claim 8, wherein the inducible promoter of the recombinant expression construct is operably linked to a reporter gene.

10. The method of claim 9, wherein the reporter gene is firefly luciferase, chloramphenicol acetyltransferase, beta-galactosidase, green fluorescent protein, or alkaline phosphatase.

11. A method for identifying a compound that decreases angiotensin-induced gene expression from a recombinant expression construct according to claim 1, comprising the steps of:
a) contacting a recombinant mammalian cell comprising said recombinant expression construct with the compound in the presence and absence of angiotensin;
b) comparing gene expression from the recombinant expression construct in the presence and absence of the compound; and
c) identifying a compound that decreases angiotensin-induced gene expression from a recombinant expression construct when gene expression is lower in the presence than in the absence of the compound.

12. The method of claim 11, wherein the inducible promoter of the recombinant expression construct is operably linked to a reporter gene.

13. The method of claim 12, wherein the reporter gene is firefly luciferase, chloramphenicol acetyltransferase, beta-galactosidase, green fluorescent protein, or alkaline phosphatase.

14. A method for reducing blood pressure in a patient comprising the step of inhibiting MEK activity in the patient.

15. The method of claim 14 wherein the patient has hypertension.

16. A method for reducing blood pressure in a patient comprising the step of inhibiting MLCK activity in the patient.

17. The method of claim 16 wherein the patient has hypertension.

18. A method for inhibiting cell proliferation comprising the step of contacting a cell with an MLCK inhibitor in combination with a chemotherapeutic agent.

19. The method of claim 18, wherein the cell is a tumor cell.

20. The method of claim 18, wherein the cell is a vascular smooth muscle cell.

21. A method for treating or preventing tumor cell growth in a patient comprising administering an effective amount of a MLCK inhibitor in combination with a chemotherapeutic agent to a patient in need thereof.

22. A method for diagnosing a proliferative disorder, comprising assaying MLCK expression in a patient sample, wherein overexpression of MLCK compared with expression of MLCK in a control sample indicates a proliferative disorder.

23. The method of claim 22, wherein the proliferative disorder is hypertension.

24. The method of claim 22, wherein the patient sample comprises vascular smooth muscle cells.

25. A method for diagnosing a proliferative disorder, comprising detecting mutations in an MLCK promoter in a patient sample.

26. The method of claim 25, wherein the mutation comprises a 12 basepair insertion having the sequence CTCTCTCTCTCT (SEQ ID NO: 1) inserted proximal to a CArG site.

27. The method of claim 25, wherein the proliferative disorder is hypertension.

28. The method of claim 25, wherein the patient sample comprises vascular smooth muscle cells.


SEQUENCE LISTING (16 sequences)

SEQ ID NO: 1 - DNA, 12 bp, Rattus rattus
ctctctctctct

SEQ ID NO: 2 - DNA, 26 bp, Artificial
  Synthetic
gtccttcaggtcgtcttctgatacgg

SEQ ID NO: 3 - DNA, 24 bp, Artificial
  Synthetic
tcaggaaccgggttggcgaatgca

SEQ ID NO: 4 - DNA, 24 bp, Artificial
  Synthetic
tgcattcgccaacccggttcctga

SEQ ID NO: 5 - DNA, 24 bp, Artificial
  Synthetic
tcaggaaccgggttggcgaatgca

SEQ ID NO: 6 - DNA, 24 bp, Artificial
  Synthetic
gtcttctgatacggtctttgtgct

SEQ ID NO: 7 - DNA, 508 bp, Rattus rattus
ggtaccgagctcggatccactagtaacggccgccagtgtgctggaattcgcccttaagcctagccaggtctcccacactcacccgccatcctctgcctggccctgaagcctggggttgtttgtgtcctcctgttaagtctgccagagccaaagagaaagattggaaagggatgaaggggagaggccagaagaaggcaaaggagggggtgatgtgtgtttctttttcctctctctctctctctctctctctctctctctctctctctctctttgcacacacccttataaggctactgaaatcattaccgatataataaaccaactaggcaagtgagtccctctcaccaatgttcccttctataaacacagcctggccggcccccacgtcctcttcccccctcctctctaccctccgccctcagggatttgctctcccctgacgtttctgtcttacccagccccttttactccctgctactttctctttttccttcactggttattgcag

SEQ ID NO: 8 - DNA, 520 bp, Rattus rattus
ggtaccgagctcggatccactagtaacggccgccagtgtgctggaattcgcccttaagcctagccaggtctcccacactcacccgccatcctctgtctggccctgaagcctggggttgtttgtgtcctcctgttaagtctgccagagccaaagagaaagattggaaagggatgaaggggagaggccagaagaaggcaaaggagggggtgatgtgtgtttctttttcctctctctctctctctctctctctctctctctctctctctctctctctctctctctttgcacacacccttataaggctactgaaatcattaccgatataataaaccaactaggcaagtgagtccctctcaccaatgttcccttctataaacacagcctggccggcccccacgtccccttcccccctcctctctaccctccgccctcagggatttgctctcccctgacgtttctgtcttacccagccccttttcctccctgctactttctctttttccttcactggttattgcag

SEQ ID NO: 9 - DNA, 45 bp, Rattus rattus
ctctctctctctctctctctctctttgcacacacccttataaggc

SEQ ID NO: 10 - DNA, 57 bp, Rattus rattus
ctctctctctctctctctctctctctctctctctctttgcacacacccttataaggc

SEQ ID NO: 11 - DNA, 80 bp, Artificial
  Synthetic
tgtgtgtttctttttcttctctctctctctctctctctctctctctctctctctctctctttgcacacacccttataagg

SEQ ID NO: 12 - DNA, 92 bp, Artificial
  Synthetic
tgtgtgtttctttttcttctctctctctctctctctctctctctctctctctctctctctctctctctctctttgcacacacccttataagg

SEQ ID NO: 13 - DNA, 21 bp, Artificial
  Synthetic
aagcctagccaggtctcccac

SEQ ID NO: 14 - DNA, 21 bp, Artificial
  Synthetic
ctgcaataaccagtgaaggaa

SEQ ID NO: 15 - DNA, 30 bp, Artificial
  Synthetic
cagcgttttcttcagagggagtacccagag

SEQ ID NO: 16 - DNA, 30 bp, Artificial
  Synthetic
tcagaagcaaatgtgaggagcgactgatcc